(19) United States
(12) Patent Application Publication
(10) Pub. No.: US 2025/0213181 A1
(43) Pub. Date: Jul. 3, 2025

(54) SYSTEMS AND METHOD FOR DETERMINING A POSITIONAL SLEEP DISORDERED BREATHING STATUS

(71) Applicants: ResMed Pty Ltd, Bella Vista, New South Wales (AU); RESMED SENSOR TECHNOLOGIES LIMITED, Dublin (IE)

(72) Inventors: Liam HOLLEY, Bella Vista, New South Wales (AU); Aoibhe Jacqueline TURNER-HEANEY, Sandyford, Dublin (IE)

(21) Appl. No.: 18/852,342

(22) PCT Filed: Mar. 29, 2023
(86) PCT No.: PCT/IB2023/053147

§ 371 Date: Sep. 27, 2024

Related U.S. Application Data
(60) Provisional application No. 63/362,164, filed on Mar. 30, 2022.

(51) Int. Cl. A61B 5/00; A61B 5/087; A61F 5/56; A61M 16/00

(52) U.S. Cl. CPC A61B 5/4818; A61B 5/087; A61B 5/4836; A61B 5/7246; A61B 5/7267; A61B 5/7282; A61F 5/56; A61M 16/024; A61M 2016/0033; A61M 2205/3375; A61M 2230/06; A61M 2230/18; A61M 2230/42; A61M 2230/62; A61M 2230/63

(57) ABSTRACT

A method and system for determining a positional sleep disordered breathing (pSDB) status associated with a respiratory device user is disclosed. Airflow data associated with the user is received. The airflow data is analyzed to identify a first time period of suspected arousal and a second time period of suspected arousal. A first time section between the identified first time period and the identified second time period is determined. The airflow data associated with the determined first time section is analyzed to identify (i) an indication of one or more respiratory events and/or (ii) an indication of one or more therapy events. Based at least in part on the (i) identified indication of one or more respiratory events and/or (ii) identified indication of one or more therapy events, the pSDB status of the user is determined, where the pSDB status is indicative of whether or not the user has pSDB.

### CROSS-REFERENCE TO RELATED APPLICATIONS

This application claims the benefit of, and priority to, U.S. Provisional Patent Application No. 63/362,164 filed on Mar. 30, 2022, which is hereby incorporated by reference herein in its entirety.

### TECHNICAL FIELD

The present disclosure relates generally to systems and methods for sleep monitoring, and more particularly, to systems and methods for determining a positional sleep disordered breathing (pSDB) status associated with a user.

### BACKGROUND

Many individuals suffer from sleep-related and/or respiratory disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (characterized by, for example, difficult in initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These individuals are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing and/or fail to perceive the benefits associated with using the system. As a result, some users will elect not to begin using the respiratory therapy system or discontinue use of the respiratory therapy system absent a demonstration of the severity of their symptoms when respiratory therapy treatment is not used. In addition, some individuals not using the respiratory therapy system may not realize that they suffer from one or more sleep-related and/or respiratory-related disorders. Furthermore, some users may only suffer from certain symptoms when sleeping in a specific body position and thus it is desirable to detect a disorder or symptoms which are associated with a particular body position.

The present disclosure is directed to solving these and other problems.

### SUMMARY

According to some implementations of the present disclosure, a method and system for determining a positional sleep disordered breathing (pSDB) status associated with a user of a respiratory therapy device is disclosed as follows. Airflow data associated with the user of the respiratory device is received. The airflow data is analyzed to identify a first time period of suspected arousal and a second time period of suspected arousal. A first time section between the identified first time period and the identified second time period is determined. The airflow data associated with the determined first time section is analyzed to identify (i) an indication of one or more respiratory events, (ii) an indication of one or more therapy events, or (iii) both (i) and (ii). Based at least in part on the (i) identified indication of one or more respiratory events, (ii) identified indication of one or more therapy events, or (iii) both (i) and (ii), the pSDB status of the user is determined, where the pSDB status is indicative of whether or not the user has pSDB.

According to some implementations of the present disclosure, a method and system for determining a pSDB status associated with a user is disclosed as follows. Sensor data associated with the user is received. Such sensor data may include airflow data as described above and later herein. The sensor data is analyzed to identify a first time period of suspected arousal and a second time period of suspected arousal. A first time section between the identified first time period and the identified second time period is determined. The sensor data associated with the determined first time section is analyzed to identify an indication of one or more respiratory events. Based at least in part on the identified indication of one or more respiratory events, the pSDB status of the user is determined, where the pSDB status is indicative of whether or not the user has pSDB.

According to some implementations of the present disclosure, a system for determining a pSDB status is disclosed. The system includes a control system configured to implement any of the methods disclosed above.

According to some implementations of the present disclosure, a system includes a control system and a memory. The control system includes one or more processors. The memory has stored thereon machine readable instructions. The control system is coupled to the memory. Any one of the methods disclosed herein is implemented when the machine executable instructions in the memory are executed by at least one of the one or more processors of the control system.

According to some implementations of the present disclosure, a computer program product comprising instructions which, when executed by a computer, cause the computer to carry out any one of the methods disclosed herein. In some implementations, the computer program product is a non-transitory computer readable medium.

According to some implementations of the present disclosure, a system includes a respiratory therapy device, a memory storing machine-readable instructions, and a control system. The respiratory therapy device is configured to supply pressurized air to a user. The control system includes one or more processors configured to execute the machine-readable instructions to receive airflow data associated with the user of the respiratory device. The control system is further configured to analyze the airflow data to identify a first time period of suspected arousal and a second time period of suspected arousal. The control system is further configured to determine a time section between the identified first time period and the identified second time period. The control system is further configured to analyze the airflow data associated with the determined time section to identify (1) an indication of one or more respiratory events, (ii) an indication of one or more therapy events, or (iii) both (i) and (ii). Based at least in part on the (i) identified one or more respiratory events, (ii) identified indication of one or more therapy events, or (iii) both (i) and (ii), the control system is further configured to determine a positional sleep disordered breathing (pSDB) status of the user, where the pSDB status is indicative of whether or not the user has pSDB.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system for determining a positional sleep disordered breathing (pSDB) status associated with a user, according to some implementations of the present disclosure.

FIG. 2 is a perspective view of at least a portion of the system of FIG. 1, a user, and a bed partner, according to some implementations of the present disclosure.

FIG. 3 illustrates a flow diagram for a method for determining a pSDB status using airflow data, according to some implementations of the present disclosure.

FIG. 4 illustrates a flow diagram for a method for determining a pSDB status using sensor data, according to some implementations of the present disclosure.

FIG. 5A illustrates the average heart rate pre-arousal and post-arousal for a first user, according to some implementations of the present disclosure.

FIG. 5B illustrates the average heart rate pre-arousal and post-arousal for a second user, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration.

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders. Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. Central Sleep Apnea (CSA) is another form of sleep disordered breathing. CSA results when the brain temporarily stops sending signals to the muscles that control breathing. Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration. A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events must fulfil both of the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is a further form of SDB. CSR is a disorder of a patient's respiratory controller in which there are rhythmic alternating periods of waxing and waning ventilation known as CSR cycles. CSR is characterized by repetitive de-oxygenation and re-oxygenation of the arterial blood. OHS is defined as the combination of severe obesity and awake chronic hypercapnia, in the absence of other known causes for hypoventilation. Symptoms include dyspnea, morning headache and excessive daytime sleepiness. COPD encompasses any of a group of lower airway diseases that have certain characteristics in common, such as increased resistance to air movement, extended expiratory phase of respiration, and loss of the normal elasticity of the lung. NMD encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

Many of these disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that can occur when the individual is sleeping.

Individuals with diabetes who also use a respiratory therapy system (for example to treat SDB) can experience positive and/or negative interactions. For example, the use of the respiratory therapy system can impact the efficacy of the individual's diabetes treatment plan (which could include a diabetes medication plan, a diet plan, an exercise plan, etc.). The impact on the efficacy of the individual's diabetes treatment plan can be positive or negative, and thus it can be difficult for these individuals to use a respiratory therapy system in adherence with a respiratory therapy plan, while also adhering to a diabetes treatment plan that remains effective. Thus, it is advantageous to monitor these individuals, and to make various adjustments to their diabetes treatment plans and their use of respiratory therapy systems in order to mitigate, optimize, etc. any interactions between their diabetes treatment plan and their respiratory therapy plan

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

Everyone has their own preferences for sleeping. Whether it's sleeping completely flat (e.g., in a horizontal position), reclined, or sitting upright; or whether it's lying on their stomach (e.g., in a prone position), on their back (in a supine position), or on the left or right side.

Breathing conditions for an individual's body are different when the individual is lying down as compared to when the individual is standing up. When the individual is sitting or is on their feet, the individual's airway is pointing generally downward, leaving breathing and airflow relatively unrestricted. However, when the individual lies down to sleep, the individual's body is imposed to breathing in a substantially horizontal position, meaning that gravity is now working against the airway. Sleep apnea and snoring can occur when the muscular tissues in the upper airway (or other muscles such as the soft palate, tongue, etc.) relax and narrow the airway, and the individual's lungs get limited air to breathe via the nose or throat. While the process of breathing is the same at night, the individual's surrounding tissues can vibrate, causing the individual to snore. Sometimes relaxed muscles can cause sleep apnea because some blockage of the airway hampers breathing fully, forcing the individual to wake up in the middle of sleep. As a result, it can be beneficial for the individual to sleep in a position that best supports the individual's breathing patterns. For example, some individual may benefit from sleeping in a reclined position rather than completely horizontal relative to ground, or sleep on the right or left side rather than in the supine position.

Sleeping in the supine position can often be problematic for those who have snoring problems, breathing problems, or sleep apnea. This happens because the gravitational force enhances the capacity of the jaw, the tongue, and soft palate to drop back toward the throat. This may narrow or collapse the airways thus causing a partial or complete cessation of breathing, or other breathing difficulties, snoring, etc.

Sleeping in the prone position may seem like an alternative to the gravity issue as the downward force pulls the tongue and palate forward. While this is true to an extent, when sleeping in this position, the individual's nose and mouth can become blocked by the pillow or other bedding, which may affect the individual's breathing. Apart from this, it may also cause neck pain, cervical problems, or digestion problems, which in turn affect the individual's sleep quality.

Some studies suggest that sleeping on the side may be the most ideal position for snoring and sleep apnea sufferers. Because when the individual's body is positioned on its side during rest, the airways are more stable and less likely to collapse or restrict airflow. In this position, the individual's body, head and torso are positioned on one side (left or right), arms are under the body or a bit forward or extended, and legs are packed with one under the other or slightly staggered. While both lateral (left and right) sides are considered as good sleeping positions, for some the left lateral position may not be an ideal one. That's because while sleeping on the left side, the internal organs of the body in the thorax can face some movement. And the lungs may add more weight or pressure on the heart. This can affect the heart's function, and it can retaliate by activating the kidneys, causing an increased need for urination at night. The right side, however, puts less pressure on the vital organs, such as lungs and heart.

Sleeping on a particular side can also be ideal if a joint (often shoulder or hip) on the individual's other side is causing pain.

When an individual has sleep apnea or other breathing disorders, getting a good and peaceful sleep becomes difficult. However, choosing the right sleeping position can help the user get comfortable and at the same time help overcome or alleviate the breathing problems that the individual usually face while sleeping. Thus, according to some implementations of the present disclosure, systems and methods are provided to cause the user to change body position if they are sleeping in an undesired body or head position (e.g., supine). Positional therapy not only can provide treatment for users with mild OSA, but also for users already undergoing another therapy who could have a more comfortable and efficacious option.

Studies have also suggested that positional OSA (pOSA) patients, compared to non-pOSA patients, have a more backward positioning of the lower jaw, lower facial height, longer posterior airway space measurements, and a smaller volume of lateral pharyngeal wall tissue. Such characteristics of the pOSA patients result in a greater lateral diameter and ellipsoid shape of the upper airway. In addition, pOSA patients tend to have a smaller neck circumference. Thus, it is suggested that even though the anterior-posterior diameter in both pOSA patients and non-pOSA patients is reduced as a result of the effect of gravity in the supine position, there is sufficient preservation of airway space and avoidance of complete upper airway collapse because of the greater lateral diameter in pOSA patients. Thus, it is advantageous to predict and/or diagnose patients with pOSA, and generate treatment plans and/or adjust treatment parameters accordingly. In some implementations, the body position of the user is taken into account when making such treatment plans and/or adjusting such treatment parameters. In some implementations, one or more steps of the methods disclosed herein may be incorporated into an application that integrates prediction, screening, diagnosis, and/or therapy.

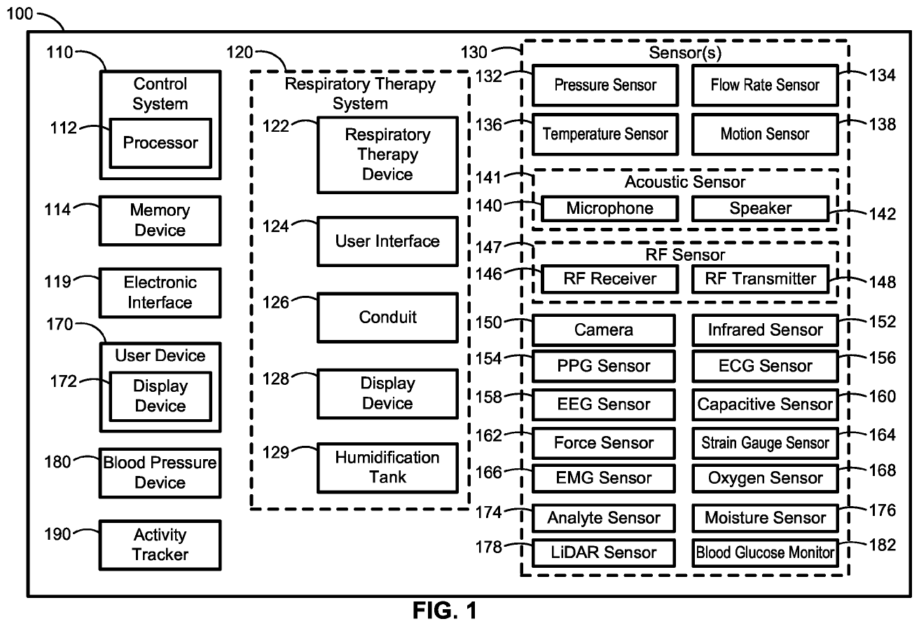

Referring to FIG. 1, a system 100, according to some implementations of the present disclosure, is illustrated. The system 100 includes a control system 110, a memory device 114, an electronic interface 119, one or more sensors 130, and optionally one or more user devices 170. In some implementations, the system 100 further includes a respiratory therapy system 120 (that includes a respiratory therapy device 122), a blood pressure device 180, an activity tracker 190, or any combination thereof. The system 100 can be used to monitor an individual who uses a respiratory therapy system and may or may not have pSDB, such as pOSA, positional CSA and other types of positional apneas, positional RERA, positional snoring, positional CSR, positional respiratory insufficiency, positional OHS, positional COPD, etc.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the user device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory therapy device 122 of the respiratory therapy system 120, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family medical history (such as a family history of insomnia or sleep apnea), an employment status of the user, an educational status of the user, a socioeconomic status of the user, or any combination thereof. The medical information can include, for example, information indicative of one or more medical conditions associated with the user, medication usage by the user, or both. The medical information data can further include a fall risk assessment associated with the user (e.g., a fall risk score using the Morse fall scale), a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological data and/or acoustic data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory therapy system 120 (also referred to as a respiratory pressure therapy system). The respiratory therapy system 120 can include a respiratory therapy device 122 (also referred to as a respiratory pressure device), a user interface 124 (also referred to as a mask or a patient interface), a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory therapy device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 120 is generally used to treat individuals suffering from one or more sleep-related respiratory disorders (e.g., obstructive sleep apnea, central sleep apnea, or mixed sleep apnea), other respiratory disorders such as COPD, or other disorders leading to respiratory insufficiency, that may manifest either during sleep or wakefulness.

The respiratory therapy device 122 is generally used to generate pressurized air that is delivered to a user (e.g., using one or more motors (such as a blower motor) that drive one or more compressors). In some implementations, the respiratory therapy device 122 generates continuous constant air pressure that is delivered to the user. In other implementations, the respiratory therapy device 122 generates two or more predetermined pressures (e.g., a first predetermined air pressure and a second predetermined air pressure). In still other implementations, the respiratory therapy device 122 is configured to generate a variety of different air pressures within a predetermined range. For example, the respiratory therapy device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory therapy device 122 can also deliver pressurized air at a predetermined flow rate between, for example, about −20 L/min and about 150 L/min, while maintaining a positive pressure (relative to the ambient pressure). In some implementations, the control system 110, the memory device 114, the electronic interface 119, or any combination thereof can be coupled to and/or positioned within a housing of the respiratory therapy device 122.

The user interface 124 engages a portion of the user's face and delivers pressurized air from the respiratory therapy device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

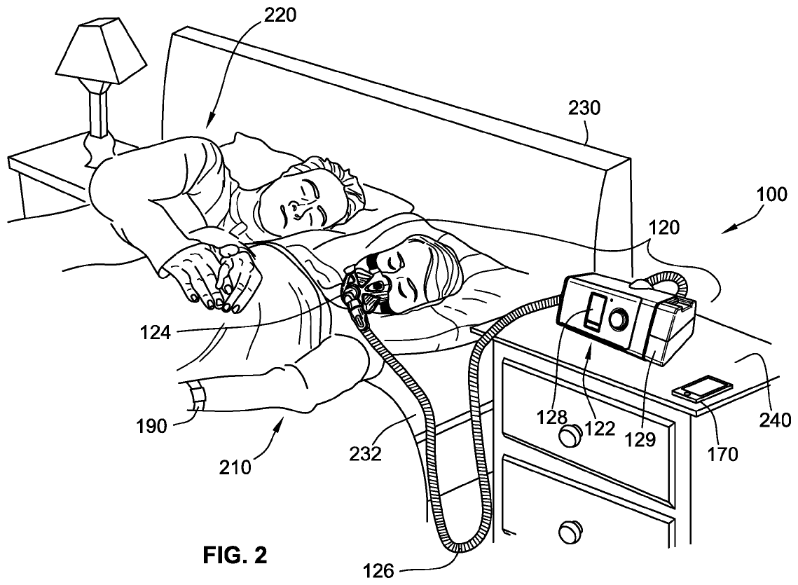

In some implementations, the user interface 124 is or includes a facial mask that covers the nose and mouth of the user (as shown, for example, in FIG. 2). Alternatively, the user interface 124 is or includes a nasal mask that provides air to the nose of the user or a nasal pillow mask that delivers air directly to the nostrils of the user. The user interface 124 can include a strap assembly that has a plurality of straps (e.g., including hook and loop fasteners) for positioning and/or stabilizing the user interface 124 on a portion of the user interface 124 on a desired location of the user (e.g., the face), and a conformal cushion (e.g., silicone, plastic, foam, etc.) that aids in providing an air-tight seal between the user interface 124 and the user.

The conduit 126 allows the flow of air between two components of a respiratory therapy system 120, such as the respiratory therapy device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user's airway. Thus, the air pathway generally includes at least a motor of the respiratory therapy device 122, the user interface 124, and the conduit 126.

One or more of the respiratory therapy device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory therapy device 122.

The display device 128 is generally used to display image(s) including still images, video images, or both and/or information regarding the respiratory therapy device 122. For example, the display device 128 can provide information regarding the status of the respiratory therapy device 122 (e.g., whether the respiratory therapy device 122 is on/off, the pressure of the air being delivered by the respiratory therapy device 122, the temperature of the air being delivered by the respiratory therapy device 122, etc.) and/or other information (e.g., a sleep score or a therapy score (such as a myAir® score, such as described in WO 2016/061629 and US 2017/0311879, each of which is hereby incorporated by reference herein in its entirety), the current date/time, personal information for the user, a questionnaire for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 122.

The humidification tank 129 is coupled to or integrated in the respiratory therapy device 122 and includes a reservoir of water that can be used to humidify the pressurized air delivered from the respiratory therapy device 122. The respiratory therapy device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

The respiratory therapy system 120 can be used, for example, as a ventilator or a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based at least in part on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory therapy system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory therapy device 122 via the conduit 126. In turn, the respiratory therapy device 122 delivers pressurized air to the user 210 via the conduit 126 and the user interface 124 to increase the air pressure in the throat of the user 210 to aid in preventing the airway from closing and/or narrowing during sleep. The respiratory therapy device 122 can include the display device 128, which can allow the user to interact with the respiratory therapy device 122. The respiratory therapy device 122 can also include the humidification tank 129, which stores the water used to humidify the pressurized air. The respiratory therapy device 122 can be positioned on a nightstand 240 that is directly adjacent to the bed 230 as shown in FIG. 2, or more generally, on any surface or structure that is generally adjacent to the bed 230 and/or the user 210. The user can also wear the blood pressure device 180 and the activity tracker 190 while lying on the mattress 232 in the bed 230.

Referring back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, an RF transmitter 148, a camera 150, an infrared (IR) sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a light detection and ranging (LiDAR) sensor 178, a blood glucose monitor 182, or any combination thereof. Generally, each of the one or sensors 130 are configured to output sensor data that is received and stored in the memory device 114 or one or more other memory devices. The sensors 130 can also include, an electrooculography (EOG) sensor, a peripheral oxygen saturation (SpO₂) sensor, a galvanic skin response (GSR) sensor, a carbon dioxide (CO₂) sensor, or any combination thereof.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the IR sensor 152, the PPG sensor 154, the ECG sensor 156, the EEG sensor 158, the capacitive sensor 160, the force sensor 162, the strain gauge sensor 164, the EMG sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate, for example physiological data, acoustic data, or both, that is associated with a user of the respiratory therapy system 120 (such as the user 210 of FIG. 2), the respiratory therapy system 120, both the user and the respiratory therapy system 120, or other entities, objects, activities, etc. Physiological data generated by one or more of the sensors 130 can be used by the control system 110 to determine a sleep-wake signal associated with the user during the sleep session and one or more sleep-related parameters. The sleep-wake signal can be indicative of one or more sleep stages (sometimes referred to as sleep states), including sleep, wakefulness, relaxed wakefulness, micro-awakenings, or distinct sleep stages such as a rapid eye movement (REM) stage (which can include both a typical REM stage and an atypical REM stage), a first non-REM stage (often referred to as “N1”), a second non-REM stage (often referred to as “N2”), a third non-REM stage (often referred to as “N3”), or any combination thereof. Methods for determining sleep stages from physiological data generated by one or more of the sensors, such as sensors 130, are described in, for example, WO 2014/047310, U.S. Pat. No. 10,492,720, U.S. Pat. No. 10,660,563, US 2020/0337634, WO 2017/132726, WO 2019/122413, US 2021/0150873, WO 2019/122414, US 2020/0383580, each of which is hereby incorporated by reference herein in its entirety. Further methods determining sleep stages from airflow data generated by one or more of the sensors, such as pressure sensor 132 and/or flow rate sensor 134, is described in WO2022/091005A1, which is hereby incorporated by reference herein in its entirety.

The sleep-wake signal can also be timestamped to indicate a time that the user enters the bed, a time that the user exits the bed, a time that the user attempts to fall asleep, etc. The sleep-wake signal can be measured one or more of the sensors 130 during the sleep session at a predetermined sampling rate, such as, for example, one sample per second, one sample per 30 seconds, one sample per minute, etc. Examples of the one or more sleep-related parameters that can be determined for the user during the sleep session based at least in part on the sleep-wake signal include a total time in bed, a total sleep time, a total wake time, a sleep onset latency, a wake-after-sleep-onset parameter, a sleep efficiency, a fragmentation index, an amount of time to fall asleep, a consistency of breathing rate, a fall asleep time, a wake time, a rate of sleep disturbances, a number of movements, or any combination thereof.

Physiological data and/or acoustic data generated by the one or more sensors 130 can also be used to determine a respiration signal associated with the user during a sleep session. The respiration signal is generally indicative of respiration or breathing of the user during the sleep session. The respiration signal can be indicative of, for example, a respiration rate, a respiration rate variability, an inspiration amplitude, an expiration amplitude, an inspiration-expiration amplitude ratio, an inspiration-expiration duration ratio, a number of events per hour, a pattern of events, pressure settings of the respiratory therapy device 122, or any combination thereof. The event(s) can include snoring, apneas, central apneas, obstructive apneas, mixed apneas, hypopneas, RERAs, a flow limitation (e.g., an event that results in the absence of the increase in flow despite an elevation in negative intrathoracic pressure indicating increased effort), a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, an elevated stress level, etc. Events can be detected by any means known in the art such as described in, for example, U.S. Pat. Nos. 5,245,995, 6,502,572, WO 2018/050913, WO 2020/104465, each of which is incorporated by reference herein in its entirety.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory therapy system 120 and/or ambient pressure. In such implementations, the pressure sensor 132 can be coupled to or integrated in the respiratory therapy device 122. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of the user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory therapy device 122, an air flow rate through the conduit 126, an air flow rate through the user interface 124, or any combination thereof. In such implementations, the flow rate sensor 134 can be coupled to or integrated in the respiratory therapy device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user, a skin temperature of the user 210, a temperature of the air flowing from the respiratory therapy device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user and proximal to the user interface 124, and configured to detect features of the pulse shape, speed, amplitude, or volume. In some implementations, the motion sensor 138 alternatively or additionally generates one or more signals representing bodily movement of the user, from which may be obtained a signal representing a sleep stage/state of the user; for example, via a respiratory movement of the user.

The microphone 140 outputs acoustic data that can be stored in the memory device 114 and/or analyzed by the processor 112 of the control system 110. The acoustic data generated by the microphone 140 is reproducible as one or more sound(s) during a sleep session (e.g., sounds from the user, sounds associated with movements of the user, components of the respiratory therapy system (e.g., the conduit), or both) to determine (e.g., using the control system 110) one or more sleep-related parameters, such as arousals of the user, as described in further detail herein. The acoustic data from the microphone 140 can also be used to identify (e.g., using the control system 110) an event experienced by the user during the sleep session, as described in further detail herein. In implementations, the acoustic data from the microphone 140 is representative of noise associated with the respiratory therapy system 120. In some implementations, the system 100 includes a plurality of microphones (e.g., two or more microphones and/or an array of microphones with beamforming) such that sound data generated by each of the plurality of microphones can be used to discriminate the sound data generated by another of the plurality of microphones. The microphone 140 can be coupled to or integrated in the respiratory therapy system 120 (or the system 100) generally in any configuration. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the user device 170 (e.g., the microphone 140 is a microphone of a smart phone). The microphone 140 can be integrated into the user interface 124, the conduit 126, the respiratory therapy device 122, or any combination thereof. In general, the microphone 140 can be located at any point within or adjacent to the air pathway of the respiratory therapy system 120, which includes at least the motor of the respiratory therapy device 122, the user interface 124, and the conduit 126. Thus, the air pathway can also be referred to as the acoustic pathway.

The speaker 142 outputs sound waves that are typically audible to the user. In one or more implementations, the sound waves can be audible to a user of the system 100 or inaudible to the user of the system (e.g., ultrasonic sound waves). The speaker 142 can be used, for example, as an alarm clock or to play an alert or message to the user (e.g., in response to an event). In some implementations, the speaker 142 can be used to communicate the acoustic data generated by the microphone 140 to the user. The speaker 142 can be coupled to or integrated in the respiratory therapy device 122, the user interface 124, the conduit 126, or the user device 170.

The microphone 140 and the speaker 142 can be used as separate devices. In some implementations, the microphone 140 and the speaker 142 can be combined into an acoustic sensor 141 (e.g., a SONAR sensor), as described in, for example, WO 2018/050913 and WO 2020/104465, each of which is hereby incorporated by reference herein in its entirety. In such implementations, the speaker 142 generates or emits sound waves at a predetermined interval and/or frequency, and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user or a bed partner of the user (such as bed partner 220 in FIG. 2). Based at least in part on the data from the microphone 140 and/or the speaker 142, the control system 110 can determine a location of the user and/or one or more of the sleep-related parameters described in herein, such as, for example, a respiration signal, a respiration rate, an inspiration amplitude, an expiration amplitude, an inspiration-expiration ratio, a number of events per hour, a pattern of events, a sleep stage, pressure settings of the respiratory therapy device 122, a mouth leak status, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user and/or one or more of the sleep-related parameters described herein. An RF receiver (either the RF receiver 146 and the RF transmitter 148 or another RF pair) can also be used for wireless communication between the control system 110, the respiratory therapy device 122, the one or more sensors 130, the user device 170, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147 (e.g., a RADAR sensor). In some such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based at least in part on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user enters the user's bed (such as bed 230 in FIG. 2), and to determine a time when the user exits the bed 230. The camera 150 can also be used to track eye movements, pupil dilation (if one or both of the user's eyes are open), blink rate, or any changes during REM sleep. The camera 150 can also be used to track the position of the user, which can impact the duration and/or severity of apneic episodes in users with positional obstructive sleep apnea.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user and/or movement of the user. The IR sensor 152 can also be used in conjunction with the camera 150 when measuring the presence, location, and/or movement of the user. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user, embedded in clothing and/or fabric that is worn by the user, embedded in and/or coupled to the user interface 124 and/or its associated headgear (e.g., straps, etc.), etc.

The ECG sensor 156 outputs physiological data associated with electrical activity of the heart of the user. In some implementations, the ECG sensor 156 includes one or more electrodes that are positioned on or around a portion of the user during the sleep session. The physiological data from the ECG sensor 156 can be used, for example, to determine one or more of the sleep-related parameters described herein.

The EEG sensor 158 outputs physiological data associated with electrical activity of the brain of the user. In some implementations, the EEG sensor 158 includes one or more electrodes that are positioned on or around the scalp of the user during the sleep session. The physiological data from the EEG sensor 158 can be used, for example, to determine a sleep stage of the user at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user. The data output by the analyte sensor 174 can be stored in the memory device 114 and used by the control system 110 to determine the identity and concentration of any analytes in the user's breath. In some implementations, the analyte sensor 174 is positioned near a mouth of the user to detect analytes in breath exhaled from the user's mouth. For example, when the user interface 124 is a facial mask that covers the nose and mouth of the user, the analyte sensor 174 can be positioned within the facial mask to monitor the user mouth breathing. In other implementations, such as when the user interface 124 is a nasal mask or a nasal pillow mask, the analyte sensor 174 can be positioned near the nose of the user to detect analytes in breath exhaled through the user's nose. In still other implementations, the analyte sensor 174 can be positioned near the user's mouth when the user interface 124 is a nasal mask or a nasal pillow mask. In this implementation, the analyte sensor 174 can be used to detect whether any air is inadvertently leaking from the user's mouth. In some implementations, the analyte sensor 174 is a volatile organic compound (VOC) sensor that can be used to detect carbon-based chemicals or compounds, such as carbon dioxide. In some implementations, the analyte sensor 174 can also be used to detect whether the user is breathing through their nose or mouth. For example, if the data output by an analyte sensor 174 positioned near the mouth of the user or within the facial mask (in implementations where the user interface 124 is a facial mask) detects the presence of an analyte, the control system 110 can use this data as an indication that the user is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user's face, near the connection between the conduit 126 and the user interface 124, near the connection between the conduit 126 and the respiratory therapy device 122, etc.). Thus, in some implementations, the moisture sensor 176 can be coupled to or integrated into the user interface 124 or in the conduit 126 to monitor the humidity of the pressurized air from the respiratory therapy device 122. In other implementations, the moisture sensor 176 is placed near any area where moisture levels need to be monitored. The moisture sensor 176 can also be used to monitor the humidity of the ambient environment surrounding the user, for example the air inside the user's bedroom. The moisture sensor 176 can also be used to track the user's biometric response to environmental changes.

One or more LiDAR sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

The blood glucose monitor 182 can be used to measure the concentration of glucose in the user's blood. The blood glucose monitor 182 can be implemented in a variety of different manners. In some implementations, the blood glucose monitor 182 is a stand-alone blood glucose monitor that analyzes blood samples (for example via optical analysis, electrochemical analysis, and/or other analysis techniques) to perform spot measurements (e.g., single point in time measurements) of the user's blood glucose. In other implementations, the blood glucose monitor 182 is a continuous glucose monitor, also referred to as a CGM. The continuous glucose monitor is able to perform continuous measurements of the user's blood glucose. In some examples, the continuous glucose monitor includes a small needle that can be inserted under the user's skin (for example the skin of the user's upper arm), that is used to continually analyze body fluid samples (e.g., blood, interstitial fluid, etc.) and measure the user's blood glucose (for example via optical analysis, electrochemical analysis, and/or other analysis techniques).

In still other implementations, the blood glucose monitor 182 can include other types of devices and/or sensors used to measure the user's blood glucose (via spot measurements and/or continuous measurements). In one example, the blood glucose monitor 182 measures blood glucose through the user's skin or other body parts (for example via optical analysis techniques such as spectroscopy, polarization measurements, etc.). In another example, blood glucose monitor 182 measures blood glucose via sweat. In a further example, the blood glucose monitor 182 measures blood glucose via their user's breath, in which case the blood glucose monitor 182 may be the same as or similar to the analyte sensor 174. Generally, the blood glucose monitor 182 can include any suitable number of blood glucose monitors. For example, in some implementations, the blood glucose monitor 182 of the system 100 may include only a device/sensor, such as a point-in-time blood glucose monitor or a continuous glucose meter. In other implementations, the blood glucose monitor 182 of the system 100 may include multiple devices and/or sensors, such as a continuous glucose meter and a device/sensor that measures the user's blood glucose via sweat analysis and/or breath analysis.

While shown separately in FIG. 1, any combination of the one or more sensors 130 can be integrated in and/or coupled to any one or more of the components of the system 100, including the respiratory therapy device 122, the user interface 124, the conduit 126, the humidification tank 129, the control system 110, the user device 170, or any combination thereof. For example, the acoustic sensor 141 and/or the RF sensor 147 can be integrated in and/or coupled to the user device 170. In such implementations, the user device 170 can be considered a secondary device that generates additional or secondary data for use by the system 100 (e.g., the control system 110) according to some aspects of the present disclosure. In some implementations, the pressure sensor 132 and/or the flow rate sensor 134 are integrated into and/or coupled to the respiratory therapy device 122. In some implementations, at least one of the one or more sensors 130 is not coupled to the respiratory therapy device 122, the control system 110, or the user device 170, and is positioned generally adjacent to the user during the sleep session (e.g., positioned on or in contact with a portion of the user, worn by the user, coupled to or positioned on the nightstand, coupled to the mattress, coupled to the ceiling, etc.). More generally, the one or more sensors 130 can be positioned at any suitable location relative to the user such that the one or more sensors 130 can generate physiological data associated with the user and/or the bed partner 220 during one or more sleep session.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, an average duration of events, a range of event durations, a ratio between the number of different events, a sleep stage, an apnea-hypopnea index (AHI), or any combination thereof. The one or more events can include snoring, apneas, central apneas, obstructive apneas, mixed apneas, hypopneas, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, hyperventilation, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The user device 170 includes a display device 172. The user device 170 can be, for example, a mobile device such as a smart phone, a tablet, a laptop, a gaming console, a smart watch, or the like. Alternatively, the user device 170 can be an external sensing system, a television (e.g., a smart television) or another smart home device (e.g., a smart speaker(s) such as Google Home™, Google Nest™, Amazon Echo™, Amazon Echo Show™®, Alexa™-enabled devices, etc.). In some implementations, the user device 170 is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 170. In some implementations, one or more user devices 170 can be used by and/or included in the system 100.

The blood pressure device 180 is generally used to aid in generating physiological data for determining one or more blood pressure measurements associated with a user. The blood pressure device 180 can include at least one of the one or more sensors 130 to measure, for example, a systolic blood pressure component and/or a diastolic blood pressure component.

In some implementations, the blood pressure device 180 is a sphygmomanometer including an inflatable cuff that can be worn by a user and a pressure sensor (e.g., the pressure sensor 132 described herein). For example, as shown in the example of FIG. 2, the blood pressure device 180 can be worn on an upper arm of the user. In such implementations where the blood pressure device 180 is a sphygmomanometer, the blood pressure device 180 also includes a pump (e.g., a manually operated bulb) for inflating the cuff. In some implementations, the blood pressure device 180 is coupled to the respiratory therapy device 122 of the respiratory therapy system 120, which in turn delivers pressurized air to inflate the cuff. More generally, the blood pressure device 180 can be communicatively coupled with, and/or physically integrated in (e.g., within a housing), the control system 110, the memory device 114, the respiratory therapy system 120, the user device 170, and/or the activity tracker 190.

The activity tracker 190 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the user, such as a smartwatch, a wristband, a ring, or a patch. For example, referring to FIG. 2, the activity tracker 190 is worn on a wrist of the user. The activity tracker 190 can also be coupled to or integrated a garment or clothing that is worn by the user. Alternatively, still, the activity tracker 190 can also be coupled to or integrated in (e.g., within the same housing) the user device 170. More generally, the activity tracker 190 can be communicatively coupled with, or physically integrated in (e.g., within a housing), the control system 110, the memory device 114, the respiratory therapy system 120, the user device 170, and/or the blood pressure device 180.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for determining a length of a conduit, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and the user device 170. As a further example, a fourth alternative system includes the control system 110, the memory device 114, the respiratory therapy system 120, at least one of the one or more sensors 130, the user device 170, and the blood pressure device 180 and/or activity tracker 190. Thus, various systems for modifying pressure settings can be formed using any portion or portions of the components shown and described herein and/or in combination with one or more other components.

Referring again to FIG. 2, in some implementations, the control system 110, the memory device 114, any of the one or more sensors 130, or a combination thereof can be located on and/or in any surface and/or structure that is generally adjacent to the bed 230 and/or the user 210. For example, in some implementations, at least one of the one or more sensors 130 can be located at a first position on and/or in one or more components of the respiratory therapy system 120 adjacent to the bed 230 and/or the user 210. The one or more sensors 130 can be coupled to the respiratory therapy system 120, the user interface 124, the conduit 126, the display device 128, the humidification tank 129, or a combination thereof.

Alternatively, or additionally, at least one of the one or more sensors 130 can be located at a second position on and/or in the bed 230 (e.g., the one or more sensors 130 are coupled to and/or integrated in the bed 230). Further, alternatively or additionally, at least one of the one or more sensors 130 can be located at a third position on and/or in the mattress 232 that is adjacent to the bed 230 and/or the user 210 (e.g., the one or more sensors 130 are coupled to and/or integrated in the mattress 232). Alternatively, or additionally, at least one of the one or more sensors 130 can be located at a fourth position on and/or in a pillow that is generally adjacent to the bed 230 and/or the user 210.

Alternatively, or additionally, at least one of the one or more sensors 130 can be located at a fifth position on and/or in the nightstand 240 that is generally adjacent to the bed 230 and/or the user 210. Alternatively, or additionally, at least one of the one or more sensors 130 can be located at a sixth position such that the at least one of the one or more sensors 130 are coupled to and/or positioned on the user 210 (e.g., the one or more sensors 130 are embedded in or coupled to fabric, clothing, and/or a smart device worn by the user 210). More generally, at least one of the one or more sensors 130 can be positioned at any suitable location relative to the user 210 such that the one or more sensors 130 can generate sensor data associated with the user 210.

In some implementations, a primary sensor, such as the microphone 140, is configured to generate acoustic data associated with the user 210 during a sleep session. The acoustic data can be based on, for example, acoustic signals in the conduit 126 of the respiratory therapy system 120. For example, one or more microphones (the same as, or similar to, the microphone 140 of FIG. 1) can be integrated in and/or coupled to (i) a circuit board of the respiratory therapy device 122, (ii) the conduit 126, (iii) a connector between components of the respiratory therapy system 120, (iv) the user interface 124, (v) a headgear (e.g., straps) associated with the user interface, or (vi) a combination thereof. In some implementations, the microphone 140 is in fluid communication with the airflow pathway (e.g., an airflow pathway between the flow generator/motor and the distal end of the conduit). By fluid communication, it is intended to also include configurations wherein the microphone is in acoustic communication with the airflow pathway without necessarily being in direct or physical contact with the airflow. For example, in some implementations, the microphone is positioned on a circuit board and in fluid communication, optionally via a duct sealed by a membrane, to the airflow pathway.

In some implementations, one or more secondary sensors may be used in addition to the primary sensor to generate additional data. In some such implementations, the one or more secondary sensors include: a microphone (e.g., the microphone 140 of the system 100), a flow rate sensor (e.g., the flow rate sensor 134 of the system 100), a pressure sensor (e.g., the pressure sensor 132 of the system 100), a temperature sensor (e.g., the temperature sensor 136 of the system 100), a camera (e.g., the camera 150 of the system 100), a vane sensor (VAF), a hot wire sensor (MAF), a cold wire sensor, a laminar flow sensor, an ultrasonic sensor, an inertial sensor, or a combination thereof.

Additionally, or alternatively, one or more microphones (the same as, or similar to, the microphone 140 of FIG. 1) can be integrated in and/or coupled to a co-locatable smart device, such as the user device 170, a TV, a watch (e.g., a mechanical watch or another smart device worn by the user), a pendant, the mattress 232, the bed 230, beddings positioned on the bed 230, the pillow, a speaker (e.g., the speaker 142 of FIG. 1), a radio, a tablet device, a waterless humidifier, or a combination thereof. A co-located smart device can be any smart device that is within range for detecting sounds emitted by the user, the respiratory therapy system 120, and/or any portion of the system 100. In some implementations, the co-located smart device is a smart device that is in the same room as the user during the sleep session.

Additionally, or alternatively, in some implementations, one or more microphones (the same as, or similar to, the microphone 140 of FIG. 1) can be remote from the system 100 (FIG. 1) and/or the user 210 (FIG. 2), so long as there is an air passage allowing acoustic signals to travel to the one or more microphones. For example, the one or more microphones can be in a different room from the room containing the system 100.

As used herein, a sleep session can be defined in a number of ways based at least in part on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the user device 170 (FIG. 1) to manually initiate or terminate the sleep session.

While the control system 110 and the memory device 114 are described and shown in FIG. 1 as being a separate and distinct component of the system 100, in some implementations, the control system 110 and/or the memory device 114 are integrated in the user device 170 and/or the respiratory therapy device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 110, the memory device 114, and at least one of the one or more sensors 130 and does not include the respiratory therapy system 120. As another example, a second alternative system includes the control system 110, the memory device 114, at least one of the one or more sensors 130, and the user device 170. As yet another example, a third alternative system includes the control system 110, the memory device 114, the respiratory therapy system 120, at least one of the one or more sensors 130, and optionally the user device 170. Thus, various systems can be formed using any portion or portions of the components shown and described herein and/or in combination with one or more other components.

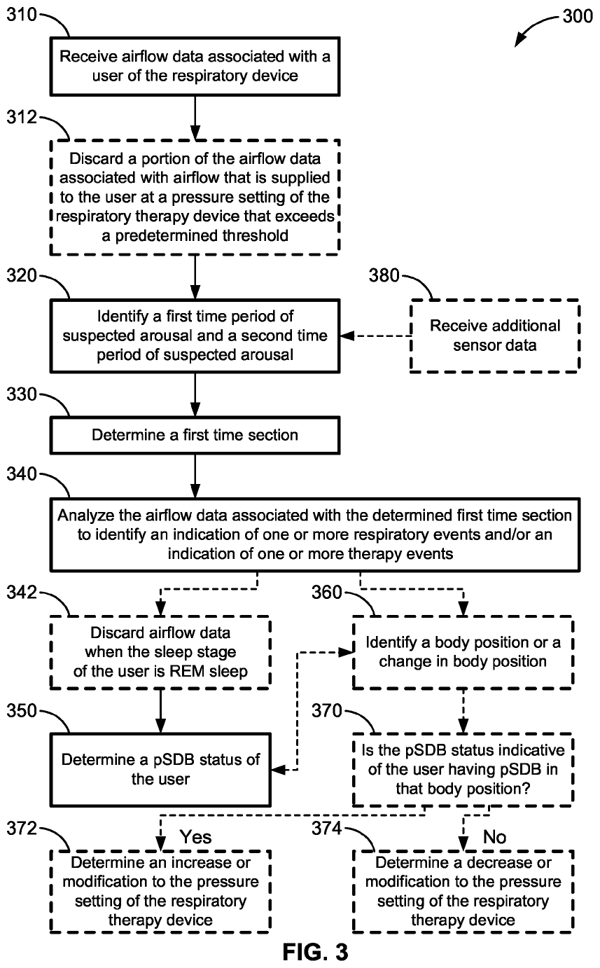

FIG. 3 illustrates a flow diagram for a method 300 for determining a pSDB status using airflow data, according to some implementations of the present disclosure. Positional obstructive sleep apnea can include position-related snoring, position-related RERAs, position-related hypopneas, positional obstructive sleep apnea, etc. The airflow data may be generated by a respiratory therapy device, such as the respiratory therapy device 122 (FIG. 1).

The method 300 begins at step 310 by receiving airflow data associated with a user of the respiratory device. The airflow data may include flow rate data associated with the respiratory therapy system, pressure data associated with the respiratory therapy system, or both.

The airflow data is analyzed, at step 320, to identify a first time period of suspected arousal and a second time period of suspected arousal. The first time period and/or the second time period may each be a point in time, a duration of time, or both. In some implementations, the suspected arousal is indicative of a body movement of the user, and is indicated by one or more features in the airflow data. In other words, the body movement of the user is inferred from a suspected arousal of the user, which arousal may be indicated by one or more features in the airflow data. In some implementations, the suspected arousal is associated with a change in body position of the user.

In some implementations, the first time period may be associated with a first movement event, and the second time period may be associated with a second movement event. In some other such implementations, the first movement event and the second movement event are different types of event.

The method 300 further provides that, at step 330, a first time section is determined between the identified first time period and the identified second time period. The method 300 further provides that, at step 340, the airflow data associated with the determined first time section is analyzed to identify an indication of one or more respiratory events and/or an indication of one or more therapy events.

For example, analyzing the airflow data associated with the user at step 340 may include processing the airflow data to identify one or more features that are indicative of the suspected arousal. Such one or more features may include an increased amplitude of flow rate signal, in increased variation in respiratory rate, a cessation of respiration, an increase in noise of the flow rate signal, an increase in the amplitude of the flow rather (e.g., corresponding to a number of larger breaths relative to an average volume of breaths) at a reduced respiratory rate (relative to a preceding and/or subsequent flow rate, e.g., an immediately preceding and/or subsequent flow rate) followed by a reduction in the amplitude of flow rate signal at an increased respiratory rate (relative to a preceding and/or subsequent flow rate, e.g., an immediately preceding and/or subsequent flow rate), or any combination thereof. For example, the cessation of respiration can be used to distinguish between holding the breath and an apnea by analyzing the duration of time (such as 1-3 seconds for holding the breath versus 10 or more seconds for experiencing an apnea).

Examples of the one or more respiratory events include a snore, a flow limitation, a residual flow limitation, a residual apnea, a residual hypopnea, a respiratory effort related arousal event (RERA) event, or any combination thereof. Examples of the identified indication of one or more respiratory events include a severity of the one or more respiratory events, a number of occurrence of the one or more respiratory events, the number of occurrence of the one or more respiratory events relative to a threshold, a type of the one or more respiratory events, an apnea-hypopnea index (AHI), or any combination thereof.

Examples of one or more therapy events include an increase in therapy pressure, a decrease in therapy pressure, a rate of change of therapy pressure, or any combination thereof. The changes in therapy pressure may be part of the AutoSet™ feature in the APAP devices. The AutoSet™ feature automatically increases therapy pressure if apnea events are detected, and reduces pressures again if a predetermined duration of time passes without an apnea event being detected The indication of one or more therapy events may include changes in therapy pressure initiated by the detection of respiratory events or predicted respiratory events. The respiratory events that would otherwise occur are prevented from doing so, and being detected or counted, by an increased pressure. As such, in some implementations, including both the respiratory events and the of therapy events may provide a more accurate assessment in relation to a user's pSDB status.

For example, step 340 may include a metric combining therapy events (e.g., change in therapy pressure) and respiratory events (e.g., a rate of respiratory events). For example, a “severity metric” may be determined using k₁P+k₂AHI, where P is therapy pressure event(s), k₁ is a coefficient associated with the therapy pressure event(s), and k₂ is a coefficient associated with the AHI, e.g., number of apnea events per hour. Such a metric may take into account the impact of the therapy pressure on the rate of events. For example, the severity metric for an AHI of 4 at 4 cmH₂O therapy pressure may be equivalent to an AHI of 1 at 10 cmH₂O. As will be understood, AHI may be replaced by any rate of events such as snore, flow limitation, etc.

Based at least in part on the identified indication of one or more respiratory events and/or the identified indication of one or more therapy events, the pSDB status of the user is determined at step 350. In some implementations, the pSDB status is indicative of whether or not the user has pSDB. In some implementations, the pSDB status includes a probability of the user having pSDB, a classification of pSDB, or both. For example, the classification of pSDB includes determining whether the pSDB is positional obstructive sleep apnea (pOSA), positional central sleep apnea (pCSA), positional respiratory effort related arousal (RERA), positional hypopneas, positional snoring, or any combination thereof.

In some implementations, the airflow data associated with the determined first time section is further analyzed, at step 360 to identify a body position or a change in body position. In some such implementations, the pSDB status is indicative of whether or not the user has pSDB in the identified body position.

In some implementations, body position may be identified based on the airflow data using a machine learning model. Examples of input for the machine learning model include patterns in the flow waveform, the shape of flow-limited breaths, the duration of expiration, determined from the airflow data. Examples of output for the machine learning model include a body position or a change in body position, or a likelihood of a body position or a change in body position. The machine learning model may have been trained using historical airflow data and reference data, wherein the reference data may include data indicative of body position and/or change in body position. For example, in some implementations, the reference data includes accelerometer data, observer scored data, or both. As another example, in some implementations, PAP pressure data, sleep staging (to allow for potential REM dominant sleep apnea as described further below), and scored airflow data (e.g., scored to highlight the respiratory events and/or body position or change in body position) may be used to train the machine learning model.

In this example, features such as respiratory rate, residual AHI residual index of other events, apnea index (AI), hypopnea index (HI), percentage of breaths with snore, percentage of breaths with flow limitation, the type of flow limitation (e.g., an identifiable shape to the inspiratory flow waveform), the duration of expiration and/or inspiration, therapy pressure (e.g., average, peak, median, and/or range), estimate of sleep state (e.g. REM, non-REM, etc.), and others, may be used to train any of the machine learning models, such as, but not limited to, support vector machine, convolutional neural network, etc.

In some implementations, the method 300 further determines, at step 370, whether the body position determined at step 360 is associated with the pSDB status that is indicative of the user having pSDB in the identified body position. In some such implementations, an increase or a modification to a pressure setting of the respiratory therapy device is determined at step 372, when the user is in the identified body position if the pSDB status is indicative of the user having pSDB in the identified body position.

In some examples, the pressure supplied to the user is increased incrementally. In some such examples, steps of the method 300 are repeated until a maximally necessary pressure limit is reached for the identified body position. For example, at some point, the pressure supplied to the user will be so high for the user that it is unlikely that the user will experience any respiratory events. As such, the maximally necessary pressure limit is associated with the highest pressure limit beyond which there is no any additional improvement to the user's respiratory events. In other words, the maximally necessary pressure limit is associated with the highest pressure at which a user's SDB is adequately treated (such that severity is at or below predetermined threshold, e.g., AHI is less than 5, 4, 3, 2, or 1, or within threshold range, e.g., AHI is 0 to 5, or 1 to 5, for example) and beyond which no additional benefit is gained.

In some implementations, a decrease or a modification to the pressure setting of the respiratory therapy device is determined at step 374, when the user is in the identified body position if the pSDB status is indicative of the user not having pSDB in the identified body position. In some examples, the pressure supplied to the user is decreased incrementally. In some such examples, steps of the method 300 are repeated until a minimally necessary pressure limit is reached for the identified body position. In some such examples, minimally necessary pressure limit is associated with the lowest pressure limit before the user begins to experience respiratory events indicative of OSA at that body position. In other words, the minimally necessary pressure limit is associated with the lowest pressure at which a user's SDB is adequately treated (such that severity is at or below predetermined threshold, e.g., AHI is less than 5, 4, 3, 2, or 1, or within threshold range, e.g., AHI is 0 to 5, or 1 to 5, for example) and below which SDB events occurs or occur such that severity is at or above the predetermined threshold or above the threshold range.

In some implementations, before analyzing to identify the first time period of suspected arousal and the second time period of suspected arousal at step 320, a portion of the airflow data is discarded at step 312, where the portion of the airflow data is associated with airflow that is supplied to the user at a pressure setting of the respiratory therapy device that exceeds a predetermined threshold. In some such implementations, the predetermined threshold is associated with a maximum therapy pressure which would suppress any pSDB events. In other words, in these implementations, the method 300 only analyzes data from lower pressures in which pSDB events are more likely to be detectable. For example, in some such implementations, the predetermined threshold is about 8 cmH₂O, about 9 cmH₂O, about 10 cmH₂O, about 11 cmH₂O, or about 12 cmH₂O. Additionally or alternatively, in some such implementations, the predetermined threshold is a percentage threshold of the user's maximum pressure as determined by the medical provider (e.g., the user's physician).

In some implementations, therapy adjustments could be made after some time into a first time section—or some time into a later time section that we learn resembles a particular type of time section determined at step 330. For example, in some such implementations, patterns associated with supine position may be identified early in a therapy session, or during previous therapy sessions, and then supine features can be identified and relied upon to modify the therapy, such as modifying the pressure response parameters.

In some implementations, a portion of the airflow data may be discarded during and after the first and second time periods of suspected arousal are determined. For example, in some such implementations, after the first time section is determined at step 330, airflow data within the first time section is analyzed and then discarded if (i) it is over a pressure threshold, (ii) REM sleep stage is detected, or (iii) both (i) and (ii).

In some implementations, heart rate data associated with the user of the respiratory device is also received at step 380. The received heart rate data may be analyzed to identify or confirm the suspected arousal. For example, the analyzing the received heart rate data can include determining a heart rate, a change in heart rate, or both, and the suspected arousal is confirmed by analyzing the determined heart rate, the determined change in heart rate, or both. In some such examples, an increase in the heart rate is indicative of suspected arousal.

The heart rate (e.g., cardiogenic activity) can also indicate arousal, such as what is described in U.S. Publication No. 2008/0045813, U.S. Publication No. 2015/0182713 A1, and WO 2005/079897, each of which is incorporated herein by reference in its entirety. Heart rate changes may occur upon arousal; therefore, by using a heart rate sensor, arousals can be identified. In some implementations, when detecting an increase in the signal noise of either the heart rate signal or the respiratory signal, movement might be inferred due to movement artifacts in the signals. Heart rate changes have also been found to correlate with the different sleep stages. For example, during REM sleep, in particular, heart rate variability is greater than other sleep stages.

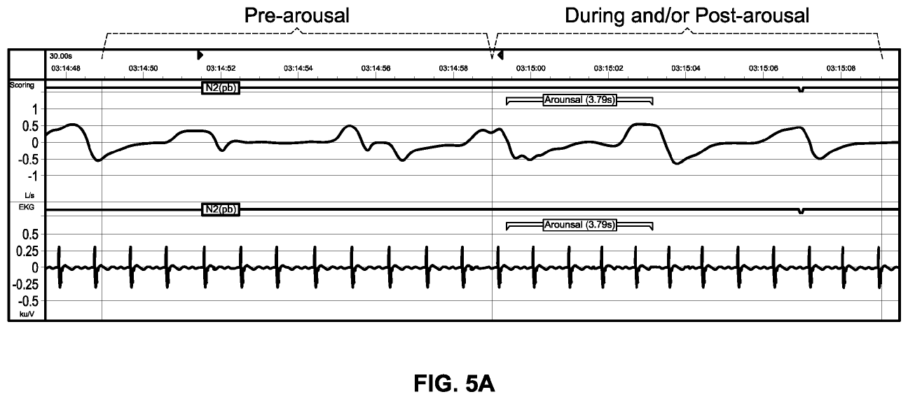

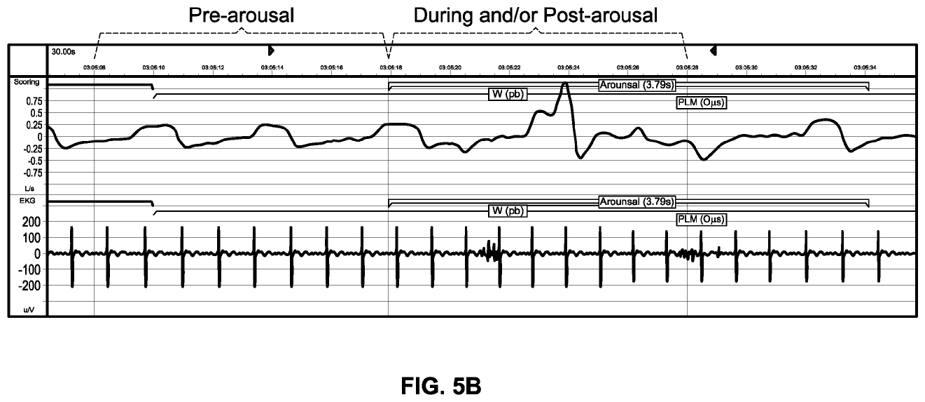

Referring briefly to FIGS. 5A-5B, FIG. 5A illustrates the average heart rate pre-arousal and post-arousal for a first user, and FIG. 5B illustrates the average heart rate pre-arousal and post-arousal for a second user, according to some implementations of the present disclosure. As shown, both users' heart rates increased upon arousal.

Turning back to FIG. 3, in some implementations, in addition to or instead of receiving heart rate data, acoustic data associated with the user of the respiratory device is received at step 380. The received acoustic data is analyzed to further determine or confirm the suspected arousal. In some implementations, the received acoustic data associated with the determined first time section is analyzed to identify a location of obstruction associated with the user if the pSDB status is indicative of the user having pSDB. For example, the location may be a point along an airway of user and/or a distance from a user interface worn by the user of the respiratory therapy device. In some such implementations, the acoustic data includes acoustic reflections of an airway of the user, an inside of mouth of the user, or both. For example, the acoustic reflections may be represented by an acoustic impedance or a distance of the acoustic impedance. Use of acoustic data to identify physical features or obstructions, such as in the airway of a respiratory therapy system user, is described in, PCT/IB2021/053603, which is incorporated in its entirety herein.

Acoustic data may additionally or alternatively be used to detect movements indicative of arousal. For example, the combination of no respiratory flow, as detected by e.g., a flow sensor, and noisy signal, as detected by e.g., a microphone, may indicate that the user is holding their breath while rolling over, whereas noise alone may indicate other forms of movement.

In some implementations, the airflow data received at step 310 is analyzed to identify (i) one or more sleep stages of the user and (ii) the one or more respiratory events experienced by the user. The one or more sleep stages of the user is correlated with the one or more respiratory events experienced by the user, to determine whether the user is experiencing rapid eye movement (REM)-dominant respiratory events.

In some such implementations, the airflow data associated with the determined first time section is further analyzed, at step 340, to identify a sleep stage of the user. The airflow data associated with the determined first time section and when the identified sleep stage of the user is REM sleep is discarded at step 342. Discarding such data ensures that REM-dominant respiratory events are not confused for pSDB-related events. Additionally or alternatively, the sleep stage data associated with the user during the therapy session is received from another source (i.e., not the airflow data received at step 310) such as a wearable sensor, sonar or radar sensor, etc. The sleep stage is determined based at least in part on the sleep stage data, and the pSDB status is associated with the sleep stage. The sleep stage includes awake, drowsy, asleep, light sleep, deep sleep, N1 sleep, N2 sleep, N3 sleep, REM sleep, or a combination thereof.

In some implementations, the method 400 further includes providing control signals to the respiratory device. Responsive to the pSDB status determined at step 350, a modification to pressure settings of the respiratory device is determined. In some implementations, the method 400 further includes providing control signals to a smart pillow. Responsive to the pSDB status determined at step 350, the smart pillow maybe adjusted such that the smart pillow urges the user to change position of the user's head. In some implementations, the method 400 further includes providing control signals to a smart bed or a smart mattress. As will be understood, a “smart” pillow, a “smart” bed or a “smart” mattress refers to an adjustable pillow, bed or mattress, respectively. The adjustable pillow, bed or mattress may be wired or wirelessly connected to and/or controlled by a user device or other such device for providing signals to the pillow, bed or mattress based on input or actions by a user, or may be automatically adjusted based on sensed data, e.g., data related to body position or change in body position of the user, data related to SDB events experienced by the user, etc. Responsive to the pSDB status determined at step 350, the smart bed or the smart mattress may be adjusted such that the smart bed or the smart mattress urges the user to change position of the user's body. In some implementations, the method 400 further includes providing control signals to a wearable device. The wearable device is couplable to a body part of the user, and responsive to the pSDB status determined at step 350, the wearable device may be adjusted such that the wearable device stimulates the user to change position of the user's body.

In some implementations, responsive to the pSDB status determined at step 350, a notification is provided to the user or a third party (e.g., a physician, home medical equipment provider (HME), etc.) via an electronic device, such that the user is alerted of the pSDB status. In some such implementations, the electronic device is an electronic display device and the providing the notification includes displaying, on the electronic display device, a message. Additionally or alternatively, the electronic device includes a speaker and the providing the notification includes playing, via the speaker, sound. In some such implementations, the sound is an alarm to wake up the user. Additionally or alternatively, the electronic device includes a haptic device worn by and/or in contact with the user, and responsive to the pSDB status determined at step 350, the haptic device urges the user to change position.

In some implementations, the frequency of the sound or vibration being transmitted may ramp up if it is detected that the user has not changed body position. In some implementations, the frequency of the sound or vibration being transmitted is adjusted proportionally to the sleep stage of the user. For example, if lightly sleeping, the stimulus may wake the user up. In some implementations, the prompt for the user to change body position requires one or both of the following conditions to be met: (i) the user is in a body position that is associated with pSDB, and (ii) one or more respiratory events such as snoring, flow limitation, hypopnea, and apnea are detected.

In implementations, the method 300 includes analyzing the breathing waveform, including the inspiratory waveform and/or expiratory waveform, and determining a deviation from a normal breathing waveform. A normal breathing waveform may be understood in terms of, for example, a model of respiratory flow, for example a numerical model, such as a half sine wave scaled in amplitude and length to fit the inspiratory (or expiratory) period and amplitude of a particular breath of a particular user, or the average of a number of breaths. In some implementations, the normal breathing waveform might be learnt for a particular user, such as a breath or average of a number of breaths during a period when the patient is determined to have good airway patency, such as during a period of wakefulness, during a period when the user is in particular sleep stage, during a period when the user is in a particular body orientation, or during a period when respiratory signals lack any indication of airway obstruction, such as flow limitation, snore, or apneas or hypopneas. As such, as will be understood, the normal breathing waveform may take the form of, for example, a curve or function such that respiratory flow can be represented as a function of time, and the deviation between a user's inspiratory breath flow and the normal waveform may be defined by any known methods of quantifying the fit between two functions or curves. For example, this could be quantified by the root mean square (RMS) error, where the greater the error, the greater the deviation. Alternatively, the deviation might be quantified as a volume of air, such as the volume of air inspired by the user over a normal inspiration volume, represented as the area between the two curves. In some cases, it may be desirable to normalize the deviation to the volume of the user's breath. For example, the deviation may be represented as the volume between the curves, as a percentage of the inspiratory volume. Thus, in certain implementations, the method 300 can include a step of fitting half a sine wave to the inspiratory waveform. For example, a half sine wave may be fit between three points, being the two zero crossing points marking the beginning and end of inspiration, and the maximum flow value in between. A measure of the fit, such as the RMS error of the fit, may then be determined. In this way, one value for every inspiratory breath is obtained. This value can be understood as a deviation from the sine wave model of inspiration, which sine wave model of inspiration may be thought of as an approximation of a normal inspiration. As such, the method 300 can give a measure of the deviation from a normal inspiratory flow. This measure can be calculated for each patient breath, and tracked for a number of breaths, or throughout a sleep and/or therapy session, or over a number of sessions, or even tracked over a longer term to determine longitudinal changes in respiration, such as that caused by disease development including, for example, respiratory diseases such as development or worsening of bronchitis, development or worsening COPD, or a COPD exacerbation, development or worsening SBD (such as OSA) or other sleep and/or respiratory conditions. In some instances, step changes in the measure of the fit, such as the RMS error of the fit, may be used as an indication of a suspected arousal and/or a change in body position. Additionally or alternatively, such step changes may be used as an indication of a change in sleep state. Such step changes can include exceeding a threshold value, such as 5, 10, 20, or 30 percent respiratory volume. In some implementations, the threshold value may be dynamically adjusted, to account for baseline breath by breath variation, for example the threshold may be set at a number of standard deviations of the deviation of a number of breaths. Alternatively, or additionally, the running deviation metric may be low pass filtered, for example with a moving average filter, to remove some of the breath by breath noise, and a step change in deviation may be assessed according to exceeding a threshold value in the low pass filtered signal. In this way, the identification of a step change is less likely to be triggered by an outlier event, such as a lone cough or sneeze. In some cases, it may be desirable to track the breath by breath variation in the deviation according to statistical properties, such as a running standard deviation, to identify periods of relative stability or instability of breathing. In other instances, oscillations in the measure of the fit, such as the RMS error of the fit, may indicate oscillations in respiratory control, and may be more sensitive than alternative parameters such as flow amplitude or ventilation volume, or minute ventilation. Such oscillations in respiratory control may comprise, for example, oscillations in respiratory drive, producing fluctuation in either amplitude or rate of respiratory effort, and hence oscillation in breath amplitude or rate.

In some implementations, it may be desirable to classify the deviation between the normal breath model and the measured breath according to particular parameters of the breath, such as, location of inspiration peak relative to the normal breath model. For example, the peak of inspiration may appear near to, substantially before, or substantially after, the model peak value, which for a half sine wave model will be equivalent to halfway through the inspiratory time.

In some implementations, a respiratory therapy system, such as, in particular, a respiratory therapy device control loop, may automatically adjust the therapy pressure to normalize the inspiratory flow shape.

Similar steps recited above can be used to identify a second time section associated with a second pSDB status. The second time section could also be associated with a different body position than the first time section. For example, if a user experiences certain respiratory events during the first time section, but not during the second time section, then the user may be experiencing pSDB for the body position at the first time section.

To determine the second time section, the airflow data is analyzed to identify a third time period of suspected arousal and a fourth time period of suspected arousal. In some implementations, the first time period, the second time period, the third time period, and the fourth time period are different time periods. In other implementations, either the second time period is the same as the third time period, or the fourth time period is the same as the first time period. A second time section is then determined between the identified third time period and the identified fourth time period.

The airflow data associated with the determined second time section is analyzed to identify another (i) indication of one or more respiratory events, (ii) indication of one or more therapy events, or (iii) both (i) and (ii). The identified indications associated with the first time section include a first number and/or type of respiratory events, therapy events, or both. The identified indications associated with the second time section include a second number and/or type of respiratory events, therapy events, or both. In this example, the step of determining the pSDB status of the user at 350 further includes comparing the first number and/or type of respiratory events, therapy events, or both to the second number and/or type of respiratory events, therapy events, or both.

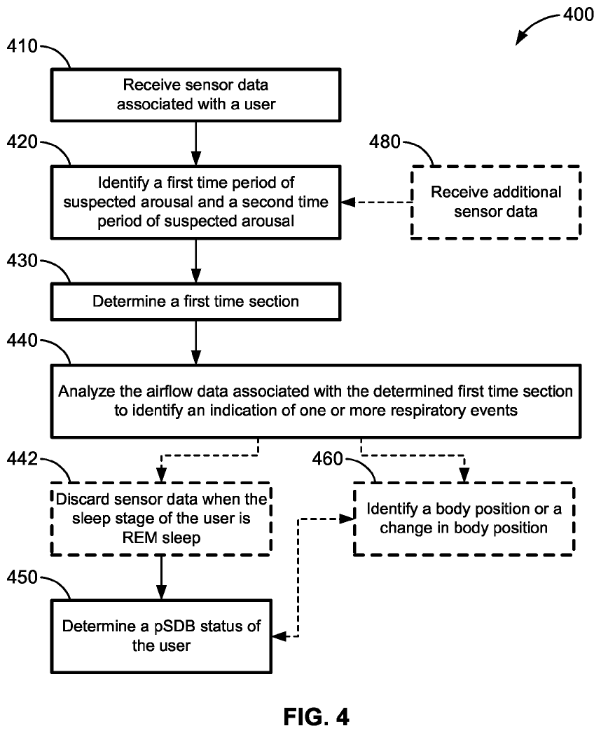

While method 300 is related to analyzing airflow data generated by the respiratory device, similar steps can be performed using devices that are not the respiratory device. For example, FIG. 4 illustrates a flow diagram for a method 400 for determining a pSDB status using sensor data. In some implementations, steps of the method 400 can be the same, or similar to, the steps of the method 300, where like reference numerals designate similar steps.

The method 400 may begin with receiving sensor data associated with the user at step 410. In some implementations, the sensor data is obtained from a motion sensor (e.g., an accelerometer). In some implementations, the motion sensor is worn on a body of the user or is an ambient sensor (e.g., radar sensor, camera, etc.) not worn by the user. In some implementations, the motion sensor is coupled to or integrated in a respiratory device of the user. In some other implementations, the motion sensor is coupled to or integrated in a mobile device. In some implementations, sensor data is received from a diagnostic device. In some such implementations, respiratory signals could be derived from a non-sealing interface of the diagnostic device (such as nasal cannula, or respiratory effort bands), from a body (e.g. chest, head, etc.) mounted accelerometer, a contact sensor (e.g., EEG, PPG, and other sensors which may be included in e.g., a smartwatch, wrist band, etc.), a non-contact sensor (such as radar, sonar, or Lidar sensors such as described herein), or acoustic sensor (such as acoustic sensor 141 as described herein, or a microphone for passive acoustic sensing, which microphone may be comprised in e.g. smart home device), or any combination thereof.

The sensor data is analyzed at step 420 to identify a first time period of suspected arousal and a second time period of suspected arousal. The suspected arousal is indicative of a body movement of the user, and indicated by one or more features in the sensor data. In some implementations, the suspected arousal is associated with a change in body position. In some implementations, the first time period is associated with a first movement event, and the second time period is associated with a second movement event. In other such implementations, the first movement event and the second movement event are the different types of event.

At step 430, a first time section between the identified first time period and the identified second time period is determined. At step 440, the sensor data associated with the determined first time section is analyzed to identify an indication of one or more respiratory events. For example, in some implementations, the analyzing the sensor data associated with the user includes processing the sensor data to identify one or more features that are indicative of the suspected arousal. The one or more respiratory events may include a snore, a flow limitation, a residual flow limitation, an apnea, a residual apnea, a hypopnea, a residual hypopnea, a respiratory effort related arousal event (RERA) event, a residual RERA event, or any combination thereof. The identified indication of one or more respiratory events may include a severity of the one or more respiratory events, a number of occurrence of the one or more respiratory events, the number of occurrence of the one or more respiratory events relative to a threshold, a type of the one or more respiratory events, an apnea-hypopnea index (AHI), or any combination thereof.

Based at least in part on the indication of one or more respiratory events identified at step 440, the pSDB status of the user is determined at step 450. The pSDB status is indicative of whether or not the user has pSDB. For example, the pSDB status may include a probability of the user having pSDB, a classification of pSDB, or both. For example, the probability of the user having pSDB can include having more severe SDB (e.g., higher AHI) when in a particular body position. For example, the classification of pSDB includes determining whether the pSDB is positional obstructive sleep apnea (pOSA), positional central sleep apnea (pCSA), positional respiratory effort related arousal (RERA), positional hypopneas, positional snoring, or any combination thereof.

In some implementations, the sensor data associated with the determined first time section is further analyzed, at step 460, to identify a body position or a change in body position. In some such implementations, the body position is identified using a machine learning model, which may be trained using historical sensor data and reference data (e.g., accelerometer data, observer scored data, or both). The reference data may include data indicative of body position and/or change in body position, for example. In some such implementations, the pSDB status determined at step 450 is further indicative of whether or not the user has pSDB in the body position identified at step 460.

In some implementations, additional sensor data, such as heart rate data and/or acoustic data, maybe received at step 480. The received additional sensor data is analyzed to determine or confirm the suspected arousal. For example, the analyzing the received heart rate data includes determining a heart rate, a change in heart rate, or both, and the suspected arousal is confirmed by analyzing the determined heart rate, the determined change in heart rate, or both. An increase in the heart rate may be indicative of suspected arousal.

In some implementations, the sensor data received at step 410 is analyzed to identify (i) one or more sleep stages of the user and (ii) the one or more respiratory events experienced by the user. The one or more sleep stages of the user is correlated with the one or more respiratory events experienced by the user to determine whether the user experiences is rapid eye movement (REM)-dominant respiratory events. The sensor data associated with the determined first time section is further analyzed at step 440 to identify a sleep stage of the user, and the sensor data associated with the determined first time section and when the identified sleep stage of the user is REM sleep is discarded at step 442.

Similar steps recited above can be used to identify a second time section associated with a second pSDB status. The second time section could also be associated with a different body position than the first time section. For example, if a user experiences certain respiratory events during the first time section, but not during the second time section, then the user may be experiencing pSDB for the body position at the first time section.

To determine the second time section, the sensor data received at step 410 is analyzed to identify a third time period of suspected arousal and a fourth time period of suspected arousal. A second time section between the identified third time period and the identified fourth time period is then determined. The sensor data associated with the determined second time section is analyzed to identify another indication of one or more respiratory events. The identified indication of one or more respiratory events associated with the first time section include a first number and/or type of respiratory events. The identified another indication of one or more respiratory events associated with the second time section include a second number and/or type of respiratory events. The step 450 of determining the pSDB status of the user further includes comparing the first number and/or type of respiratory events to the second number and/or type of respiratory events.

In some implementations, one or more steps of the methods disclosed herein may be incorporated into distributed systems for pSDB prediction, screening, diagnosis, and/or treatment. In one example, a first user device, such as a smartwatch may pick up a heart rate of the user, or any other physiological parameters as disclosed herein. For example, a separate sensor (such as an accelerometer) of the first user device on the chest and/or the head of the user may be activated to determine a torso and/or head position. An analysis is then performed to determine if the head position or the torso position or both are important for the user. In some such implementations, the user device may also be configured to generate a notification (e.g., buzz, sound, etc.) as needed to alert the user.

Generally, the methods 300 and 400 can be implemented using a system having a control system with one or more processors, and a memory storing machine readable instructions. The controls system can be coupled to the memory; the methods 300 and 400 can be implemented when the machine readable instructions are executed by at least one of the processors of the control system. The methods 300 and 400 can also be implemented using a computer program product (such as a non-transitory computer readable medium) comprising instructions that when executed by a computer, cause the computer to carry out the steps of the methods 300 and 400.

While the system 100 and the methods 300 and 400 have been described herein with reference to a single user, more generally, the system 100 and the methods 300 and 400 can be used with a plurality of users simultaneously (e.g., two users, five users, 10 users, 20 users, etc.). For example, the system 100 and methods 300 and 400 can be used in a cloud monitoring setting.

While some examples of the system 100 and the methods 300 and 400 have been described herein with reference to determining a pSDB status, more generally, the system 100 and the methods 300 and 400 can be used to determine one or more other health-related issues, such as any disease or condition that increases sympathetic activity, examples of which include COPD, CVD, somatic syndromes, etc.

In some implementations, multiple therapy modes can be combined. For example, a positional therapy can be combined with a positive airway pressure therapy, such that the pressure requirements of the positive airway pressure therapy may be reduced in certain body positions. In some implementations, a position monitoring application can be combined with a positive airway therapy, such that the user position is factored into an algorithm for determining the target therapy pressure. For example, the target therapy may be increased when the user transitions to a horizontal position, or the target pressure may be increased when the user transitions from a prone or side position (or any other position) to a supine position. Similarly, the target pressure may be reduced when the user transitions away from a supine position. In some implementations, demographic data, and/or historical therapy data may be used to estimate the magnitude in change in target pressure to be applied at a particular transition in position.

Further implementations of the disclosure include:
1. A method for determining a positional sleep disordered breathing (pSDB) status associated with a user of a respiratory therapy device, the method comprising:
   receiving airflow data associated with the user of the respiratory device;
   analyzing the airflow data to identify a first time period of suspected arousal and a second time period of suspected arousal;
   determining a first time section between the identified first time period and the identified second time period;
   analyzing the airflow data associated with the determined first time section to identify (i) an indication of one or more respiratory events, (ii) an indication of one or more therapy events, or (iii) both (i) and (ii); and
   based at least in part on the (i) identified indication of one or more respiratory events, (ii) identified indication of one or more therapy events, or (iii) both (i) and (ii), determining the pSDB status of the user, the pSDB status being indicative of whether or not the user has pSDB.
2. The method of implementation 1, wherein the airflow data includes flow rate data, pressure data, or both.
3. The method of implementation 1 or implementation 2, wherein the suspected arousal is indicated by one or more features in the airflow data.
4. The method of any one of implementations 1 to 3, wherein the suspected arousal is indicative of a body movement of the user.
5. The method of any one of implementation 1 to 4, wherein the suspected arousal is associated with a change in body position.
6. The method of any one of implementations 1 to 5, wherein the first time period is associated with a first movement event, and the second time period is associated with a second movement event.
7. The method of any one of implementations 1 to 6, wherein the one or more respiratory events include a snore, a flow limitation, a residual flow limitation, an apnea, a residual apnea, a hypopnea, a residual hypopnea, a respiratory effort related arousal event (RERA) event, or any combination thereof.
8. The method of any one of implementations 1 to 7, wherein the one or more therapy events include an increase in therapy pressure, a decrease in therapy pressure, a rate of change of therapy pressure, or any combination thereof.
9. The method of any one of implementations 1 to 8, wherein the airflow data associated with the determined first time section is further analyzed to identify a body position or a change in body position.
10. The method of implementation 9, wherein the body position is identified using a machine learning model.
11. The method of implementation 10, wherein the machine learning model is trained using historical airflow data and reference data.
12. The method of implementation 11, wherein the reference data includes accelerometer data, observer scored data, or both.
13. The method of any one of implementations 9 to 12, wherein the pSDB status is indicative of whether or not the user has pSDB in the identified body position.
14. The method of implementation 13, further comprising determining an increase or a modification to a pressure setting of the respiratory therapy device when the user is in the identified body position if the pSDB status is indicative of the user having pSDB in the identified body position.
15. The method of implementation 14, wherein steps of the method are repeated until a maximally necessary pressure limit is reached for the identified body position.
16. The method of implementation 13, further comprising determining a decrease or a modification to a pressure setting of the respiratory therapy device when the user is in the identified body position if the pSDB status is indicative of the user not having pSDB in the identified body position.
17. The method of implementation 16, wherein steps of the method are repeated until a minimally necessary pressure limit is reached for the identified body position.
18. The method of any one of implementations 1 to 17, wherein the identified indication of one or more respiratory events includes a severity of the one or more respiratory events, a number of occurrences of the one or more respiratory events, the number of occurrences of the one or more respiratory events relative to a threshold, a type of the one or more respiratory events, an apnea-hypopnea index (AHI), or any combination thereof.
19. The method of any one of implementations 1 to 18, further comprising discarding, before analyzing to identify the first time period of suspected arousal and the second time period of suspected arousal, a portion of the airflow data associated with airflow that is supplied to the user at a pressure setting of the respiratory therapy device that exceeds a predetermined threshold.
20. The method of any one of implementations 1 to 19, further comprising analyzing at least a portion of the airflow data associated with the determined first time section wherein the portion follows a decrease to a pressure setting of the respiratory therapy device, to identify (i) an indication of one or more respiratory events, (ii) an indication of one or more therapy events, or (iii) both (i) and (ii).
21. The method of any one of implementations 1 to 20, further comprising:
   analyzing the airflow data to identify a third time period of suspected arousal and a fourth time period of suspected arousal;
   determining a second time section between the identified third time period and the identified fourth time period; and
   analyzing the airflow data associated with the determined second time section to identify (i) an indication of one or more respiratory events, (ii) an indication of one or more therapy events, or (iii) both (i) and (ii), wherein a therapy pressure associated with the second time period is lower than a therapy pressure associated with the first time period.
22. The method of any one of implementations 1 to 21, further comprising discarding, before analyzing the airflow data associated with the determined first time section, a portion of the airflow data associated with airflow that is supplied to the user at a pressure setting of the respiratory therapy device that exceeds a predetermined threshold.
23. The method of implementation 19 or implementation 22, wherein the predetermined threshold is about 10 cmH₂O.
24. The method of any one of implementations 1 to 23, wherein the pSDB status includes a probability of the user having pSDB, a classification of pSDB, or both.
25. The method of implementation 24, wherein the classification of pSDB includes determining whether the pSDB is positional obstructive sleep apnea (pOSA), positional central sleep apnea (pCSA), positional respiratory effort related arousal (RERA), positional hypopneas, positional snoring, or any combination thereof.
26. The method of any one of implementations 1 to 25, further comprising:
   receiving heart rate data associated with the user of the respiratory device; and
   analyzing the received heart rate data to confirm the suspected arousal.
27. The method of implementation 26, wherein the analyzing the received heart rate data includes determining a heart rate, a change in heart rate, or both, and the suspected arousal is confirmed based on the determined heart rate, the determined change in heart rate, or both.
28. The method of implementation 27, wherein an increase in the heart rate is indicative of suspected arousal.
29. The method of any one of implementations 1 to 28, further comprising: receiving acoustic data associated with the user of the respiratory device; and analyzing the received acoustic data to identify or confirm the first time period of suspected arousal and/or the second time period of suspected arousal.
30. The method of implementation 29, wherein analyzing the received acoustic data includes detecting sounds associated with a body movement of the user and/or a change in body position of the user.
31. The method of any one of implementations 1 to 30, further comprising:
   receiving acoustic data associated with the user of the respiratory device; and
   analyzing the received acoustic data associated with the determined first time section to identify a location of obstruction associated with the user if the pSDB status is indicative of the user having pSDB.
32. The method of implementation 31, wherein the location is a point or region along an airway of user.
33. The method of implementation 31 or implementation 32, wherein the location is a distance from a user interface worn by the user of the respiratory therapy device.
34. The method of any one of implementations 31 to 33, wherein the acoustic data includes acoustic reflections of an airway of the user, an inside of mouth of the user, or both.
35. The method of implementation 34, wherein the acoustic reflections are represented by an acoustic impedance or a distance of the acoustic impedance.
36. The method of any one of implementations 1 to 35, further comprising:
   receiving acoustic data associated with the user of the respiratory device; and
   analyzing the received acoustic data to further determine or confirm the suspected arousal.
37. The method of any one of implementations 1 to 36, further comprising:
   analyzing the airflow data to identify (i) one or more sleep stages of the user and (ii) the one or more respiratory events experienced by the user; and
   correlating the one or more sleep stages of the user and the one or more respiratory events experienced by the user to determine whether the user experiences is rapid eye movement (REM)-dominant respiratory events.
38. The method of implementation 37, further comprising:
   analyzing the airflow data associated with the determined first time section to further identify a sleep stage of the user; and
   discarding the airflow data associated with the determined first time section and when the identified sleep stage of the user is REM sleep.
39. The method of any one of implementations 1 to 38, wherein the analyzing the airflow data associated with the user includes processing the airflow data to identify one or more features that are indicative of the suspected arousal.
40. The method of implementation 39, wherein the one or more features include an increased amplitude of flow rate signal, in increased variation in respiratory rate, a cessation of respiration, an increase in noise of the flow rate signal, an increase in the amplitude of flow rate at a reduced respiratory rate followed by a reduction in the amplitude of flow rate signal at a relatively increased respiratory rate, or any combination thereof.
41. The method of any one of implementations 1 to 40 wherein the analyzing the airflow data includes detecting a breathing waveform, which breathing waveform includes an inspiratory waveform and/or an expiratory waveform, and determining a deviation from a normal breathing waveform.
42. The method of implementation 41, wherein determining the deviation from the normal breathing waveform includes analyzing the user's respiratory flow as a function of time and quantifying a measure of fit between inspiratory waveform and/or an expiratory waveform and the normal breathing waveform.
43. The method of implement 41 or 42, wherein determining the deviation from the normal breathing waveform includes fitting a half sine wave to the inspiratory waveform and determining a measure of fit of the half sine wave to the inspiratory waveform.
44. The method of implementation 42, wherein the measure of fit is a root mean square (RMS) error of the fit.
45. The method of implementation 42, wherein the measure of fit describes deviation from a normal inspiratory flow.
46. The method of implementation 45, wherein the deviation from the normal inspiratory flow is monitored over one or more respiratory therapy sessions, wherein the deviation is indicative of development or worsening of a respiratory disease.
47. The method of implementation 46, wherein the respiratory disease is one or more of bronchitis, COPD, or sleep-related disorder.
48. The method of any one of implementations 42 to 47, wherein a step change of the measure of fit indicates a change in body position or sleep state of the user.
49. The method of any one of implementations 41 to 48, wherein the normal breathing waveform is determined during a period when the user is determined to have good airway patency, a period when the user is in a particular sleep stage, or a period without any indication of airway obstruction.
50. The method of any one of implementations 1 to 49, further comprising:
   providing control signals to the respiratory device; and
   responsive to the pSDB status, determining a modification to pressure settings of the respiratory device, the pressure settings being associated with pressurized air supplied to the airway of the user.
51. The method of any one of implementations 1 to 50, further comprising:
   providing control signals to a smart pillow; and
   responsive to the pSDB status, determining a modification to the smart pillow such that implementation of the modification to the smart pillow urges the user to change position of the user's head.
52. The method of any one of implementations 1 to 51, further comprising:
   providing control signals to a smart bed or a smart mattress; and
   responsive to the pSDB status, determining a modification to the smart bed or the smart mattress such that implementation of the modification to the smart bed or the smart mattress urges the user to change position of the user's body.
53. The method of any one of implementations 1 to 52, further comprising:
   providing control signals to a wearable device, the wearable device being couplable to a body part of the user; and
   responsive to the pSDB status, determining a modification to the wearable device such that implementation of the modification to the wearable device stimulates the user to change position of the user's body.
54. The method of any one of implementations 1 to 53, further comprising responsive to the pSDB status, causing a notification to be provided to the user or a third party via an electronic device, such that the user or the third party is alerted to the pSDB status.
55. The method of implementation 54, wherein the electronic device is an electronic display device and the providing the notification includes displaying, on the electronic display device, a message.
56. The method of implementation 54 or implementation 55, wherein the electronic device includes a speaker and the providing the notification includes playing, via the speaker, sound.
57. The method of implementation 56, wherein the sound is an alarm to wake up the user.
58. The method of any one of implementations 1 to 57, further comprising:
   receiving sleep stage data associated with the user during a respiratory therapy session;
   determining a sleep stage based at least in part on the sleep stage data; and
   associating the pSDB status with the sleep stage.
59. The method of implementation 58, wherein the sleep stage includes awake, drowsy, asleep, light sleep, deep sleep, N1 sleep, N2 sleep, N3 sleep, REM sleep, or a combination thereof.
60. The method of any one of implementations 1 to 59, further comprising:
   analyzing the airflow data to identify a third time period of suspected arousal and a fourth time period of suspected arousal;
   determining a second time section between the identified third time period and the identified fourth time period; and
   analyzing the airflow data associated with the determined second time section to identify another (i) indication of one or more respiratory events, (ii) indication of one or more therapy events, or (iii) both (i) and (ii),
   wherein the (i) identified indication of one or more respiratory events, (ii) identified indication of one or more therapy events, or (iii) both (i) and (ii) associated with the first time section include a first number and/or type of respiratory events, therapy events, or both,
   wherein the (i) identified another indication of one or more respiratory events, (ii) identified another indication of one or more therapy events, or (iii) both (i) and (ii) associated with the second time section include a second number and/or type of respiratory events, therapy events, or both, and
   wherein determining the pSDB status of the user includes comparing the first number and/or type of respiratory events, therapy events, or both to the second number and/or type of respiratory events, therapy events, or both.
61. The method of implementation 60, wherein the second time period is the same as the third time period.
62. The method of claim 60, wherein the fourth time period is the same as the first time period.

Still further implementations of the disclosure include:
63. A method for determining a positional sleep disordered breathing (pSDB) status associated with a user, the method comprising:
   receiving sensor data associated with the user;
   analyzing the sensor data to identify a first time period of suspected arousal and a second time period of suspected arousal;
   determining a first time section between the identified first time period and the identified second time period;
   analyzing the sensor data associated with the determined first time section to identify an indication of one or more respiratory events; and
   based at least in part on the identified indication of one or more respiratory events, determining the pSDB status of the user, the pSDB status being indicative of whether or not the user has pSDB.
64. The method of implementation 63, wherein the sensor data is obtained from one or sensors selected from a body-mounted accelerometer, a contact sensor, a non-contact sensor, an acoustic sensor, or any combination thereof.
65. The method of implementation 63 or 64, wherein the sensor data is obtained from a diagnostic device.
66. The method of implementation 65, wherein respiratory signals are derived from a non-sealing interface or respiratory effort bands of the diagnostic device.
67. The method of implementation 66, wherein the non-sealing interface is a nasal cannula.
68. The method of any one of implementations 63 to 67, wherein the sensor data is obtained from a motion sensor.
69. The method of implementation 68, wherein the motion sensor includes an accelerometer.
70. The method of implementation 68 or implementation 69, wherein the motion sensor is worn on a body of the user or an ambient sensor not worn by the user.
71. The method of implementation 68 or implementation 69, wherein the motion sensor is coupled to or integrated in a respiratory device of the user.
72. The method of implementation 68 or implementation 69, wherein the motion sensor is coupled to or integrated in a mobile device.
73. The method of any one of implementations 63 to 72, wherein respiratory signals are derived from the sensor data.
74. The method of any one of implementations 63 to 73, wherein the suspected arousal is indicated by one or more features in the sensor data.
75. The method of any one of implementations 63 to 74, wherein the suspected arousal is indicative of a body movement of the user.
76. The method of any one of implementations 63 to 75, wherein the suspected arousal is associated with a change in body position.
77. The method of any one of implementations 63 to 76, wherein the first time period is associated with a first movement event, and the second time period is associated with a second movement event.
78. The method of any one of implementations 63 to 77, wherein the one or more respiratory events include a snore, a flow limitation, a residual flow limitation, a residual apnea, a residual hypopnea, a respiratory effort related arousal event (RERA) event, or any combination thereof.
79. The method of any one of implementations 63 to 78, wherein the sensor data associated with the determined first time section is further analyzed to identify a body position or a change in body position.
80. The method of implementation 79, wherein the body position is identified using a machine learning model.
81. The method of implementation 80, wherein the machine learning model is trained using historical sensor data and reference data.
82. The method of implementation 81, wherein the reference data includes accelerometer data, observer scored data, or both.
83. The method of any one of implementations 79 to 82, wherein the pSDB status is indicative of whether or not the user has pSDB in the identified body position.
84. The method of any one of implementations 63 to 83, wherein the identified indication of one or more respiratory events includes a severity of the one or more respiratory events, a number of occurrence of the one or more respiratory events, the number of occurrence of the one or more respiratory events relative to a threshold, a type of the one or more respiratory events, an apnea-hypopnea index (AHI), or any combination thereof.
85. The method of any one of implementations 63 to 84, wherein the pSDB status includes a probability of the user having pSDB, a classification of pSDB, or both.
86. The method of implementation 85, wherein the classification of pSDB includes determining whether the pSDB is positional obstructive sleep apnea (pOSA), positional central sleep apnea (pCSA), positional respiratory effort related arousal (RERA), positional hypopneas, positional snoring, or any combination thereof.
87. The method of any one of implementations 63 to 86, further comprising:
   receiving heart rate data associated with the user; and
   analyzing the received heart rate data to determine or confirm the suspected arousal.
88. The method of implementation 87, wherein the analyzing the received heart rate data includes determining a heart rate, a change in heart rate, or both, and the suspected arousal is confirmed by analyzing the determined heart rate, the determined change in heart rate, or both.
89. The method of implementation 88, wherein an increase in the heart rate is indicative of suspected arousal.
90. The method of any one of implementations 63 to 89, further comprising: receiving acoustic data associated with the user; and analyzing the received acoustic data to determine or confirm the suspected arousal.
91. The method of any one of implementations 63 to 90, further comprising:
   analyzing the sensor data to identify (i) one or more sleep stages of the user and (ii) the one or more respiratory events experienced by the user; and
   correlating the one or more sleep stages of the user and the one or more respiratory events experienced by the user to determine whether the user experiences is rapid eye movement (REM)-dominant respiratory events.
92. The method of implementation 91, further comprising:
   analyzing the sensor data associated with the determined first time section to further identify a sleep stage of the user; and
   discarding the sensor data associated with the determined first time section and when the identified sleep stage of the user is REM sleep.
93. The method of any one of implementations 63 to 92, wherein the analyzing the sensor data associated with the user includes processing the sensor data to identify one or more features that are indicative of the suspected arousal.
94. The method of any one of implementations 63 to 92 wherein the analyzing the sensor data includes detecting a breathing waveform, which breathing waveform includes an inspiratory waveform and/or an expiratory waveform, and determining a deviation from a normal breathing waveform.
95. The method of implementation 94, wherein determining the deviation from the normal breathing waveform includes analyzing the user's respiratory flow as a function of time and quantifying a measure of fit between inspiratory waveform and/or an expiratory waveform and the normal breathing waveform.
96. The method of implementation 94 or 95, wherein determining the deviation from the normal breathing waveform includes fitting a half sine wave to the inspiratory waveform and determining a measure of fit of the half sine wave to the inspiratory waveform.
97. The method of implementation 96, wherein the measure of fit is a root mean square (RMS) error of the fit.
98. The method of implementation 96, wherein the measure of fit describes deviation from a normal inspiratory flow.
99. The method of implementation 97, wherein the deviation from the normal inspiratory flow is monitored over one or more respiratory therapy sessions, wherein the deviation is indicative of development or worsening of a respiratory disease.
100. The method of implementation 98, wherein the respiratory disease is one or more of bronchitis, COPD, or sleep-related disorder.
101. The method of implementations 96 to 100, wherein a step change of the measure of fit indicates a change in body position or sleep state of the user.
102. The method of any one of implementations 94 to 101, wherein the normal breathing waveform is determined during a period when the user is determined to have good airway patency, when the user is in a particular sleep stage, or a period without any indication of airway obstruction.
103. The method of any one of implementations 63 to 102, further comprising:
   providing control signals to a respiratory device; and
   responsive to the pSDB status, determining a modification to pressure settings of the respiratory device, the pressure settings being associated with pressurized air supplied to the airway of the user.
104. The method of any one of implementations 63 to 103 further comprising:
   providing control signals to a smart pillow; and
   responsive to the pSDB status, adjusting the smart pillow such that the smart pillow urges the user to change position of the user's head.
105. The method of any one of implementations 63 to 104, further comprising:
   providing control signals to a smart bed or a smart mattress; and
   responsive to the pSDB status, adjusting the smart bed or the smart mattress such that the smart bed or the smart mattress urges the user to change position of the user's body.
106. The method of any one of implementations 63 to 105, further comprising:
   providing control signals to a wearable device, the wearable device being couplable to a body part of the user; and
   responsive to the pSDB status, adjusting the wearable device such that the wearable device stimulates the user to change position of the user's body.
107. The method of any one of implementations 63 to 106, further comprising responsive to the pSDB status, causing a notification to be provided to the user or a third party via an electronic device, such that the user is alerted of the pSDB status.
108. The method of implementation 107, wherein the electronic device is an electronic display device and the providing the notification includes displaying, on the electronic display device, a message.
109. The method of implementation 107 or implementation 108, wherein the electronic device includes a speaker and the providing the notification includes playing, via the speaker, sound.
110. The method of implementation 109, wherein the sound is an alarm to wake up the user.
111. The method of any one of implementations 63 to 109, further comprising:
   receiving sleep stage data associated with the user during the therapy session;
   determining a sleep stage based at least in part on the sleep stage data; and
   associating the pSDB status with the sleep stage.
112. The method of implementation 111, wherein the sleep stage includes awake, drowsy, asleep, light sleep, deep sleep, N1 sleep, N2 sleep, N3 sleep, REM sleep, or a combination thereof.
113. The method of any one of implementations 63 to 112, further comprising:
   analyzing the sensor data to identify a third time period of suspected arousal and a fourth time period of suspected arousal;
   determining a second time section between the identified third time period and the identified fourth time period; and
   analyzing the sensor data associated with the determined second time section to identify another indication of one or more respiratory events,
   wherein the identified indication of one or more respiratory events associated with the first time section include a first number and/or type of respiratory events,
   wherein the identified another indication of one or more respiratory events associated with the second time section include a second number and/or type of respiratory events, and
   wherein determining the pSDB status of the user includes comparing the first number and/or type of respiratory events to the second number and/or type of respiratory events.
114. The method of implementation 113, wherein the second time period is the same as the third time period.
115. The method of implementation 113, wherein the fourth time period is the same as the first time period.

One or more elements or aspects or steps, or any portion(s) thereof, from one or more of any of the claims below can be combined with one or more elements or aspects or steps, or any portion(s) thereof, from one or more of any of the other claims or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A system for determining a positional sleep disordered breathing (pSDB) status associated with a user of a respiratory device, the system including a control system configured to:
receive airflow data associated with the user of the respiratory device;
analyze the airflow data to identify a first time period of suspected arousal and a second time period of suspected arousal;
determine a first time section between the identified first time period and the identified second time period;
analyze the airflow data associated with the determined first time section to identify (i) an indication of one or more respiratory events, (ii) an indication of one or more therapy events, or (iii) both (i) and (ii); and
based at least in part on the (i) identified indication of one or more respiratory events, (ii) identified indication of one or more therapy events, or (iii) both (i) and (ii), determine the pSDB status of the user, the pSDB status being indicative of whether or not the user has pSDB.

2. (canceled)

3. The system of claim 1, wherein the suspected arousal is indicated by one or more features in the airflow data, and wherein the suspected arousal is indicative of a body movement of the user or a change in body position.

4-6. (canceled)

7. The system of claim 1, wherein the one or more respiratory events include a snore, a flow limitation, a residual flow limitation, an apnea, a residual apnea, a hypopnea, a residual hypopnea, a respiratory effort related arousal event (RERA) event, or any combination thereof, and wherein the one or more therapy events include an increase in therapy pressure, a decrease in therapy pressure, a rate of change of therapy pressure, or any combination thereof.

8. (canceled)

9. The system of claim 1, wherein the airflow data associated with the determined first time section is further analyzed to identify a body position or a change in body position.

10. The system of claim 9, wherein the body position is identified using a machine learning model trained using historical airflow data and reference data.

11-12. (canceled)

13. The system of claim 9, wherein the pSDB status is indicative of whether or not the user has pSDB in the identified body position and wherein the control system is further configured to determine an increase or a modification to a pressure setting of the respiratory therapy device when the user is in the identified body position if the pSDB status is indicative of the user having pSDB in the identified body position and wherein the control system is further configured to determine a decrease or a modification to a pressure setting of the respiratory therapy device when the user is in the identified body position if the pSDB status is indicative of the user not having pSDB in the identified body position.

14-17. (canceled)

18. The system of claim 1, wherein the identified indication of one or more respiratory events includes a severity of the one or more respiratory events, a number of occurrences of the one or more respiratory events, the number of occurrences of the one or more respiratory events relative to a threshold, a type of the one or more respiratory events, an apnea-hypopnea index (AHI), or any combination thereof.

19-20. (canceled)

21. The system of claim 1, wherein the control system is further configured to:
analyze the airflow data to identify a third time period of suspected arousal and a fourth time period of suspected arousal;
determine a second time section between the identified third time period and the identified fourth time period; and
analyze the airflow data associated with the determined second time section to identify (i) an indication of one or more respiratory events, (ii) an indication of one or more therapy events, or (iii) both (i) and (ii), wherein a therapy pressure associated with the second time period is lower than a therapy pressure associated with the first time period.

22-24. (canceled)

25. The system of claim 1, wherein the psDB status includes a classification of pSDB including determining whether the pSDB is positional obstructive sleep apnea (pOSA), positional central sleep apnea (pCSA), positional respiratory effort related arousal (RERA), positional hypopneas, positional snoring, or any combination thereof.

26. The system of claim 1, wherein the control system is further configured to:
receive heart rate data associated with the user of the respiratory device; and
analyze the received heart rate data to confirm the suspected arousal.

27-28. (canceled)

29. The system of claim 1, wherein the control system is further configured to:
receive acoustic data associated with the user of the respiratory device; and
analyze the received acoustic data to identify or confirm the first time period of suspected arousal and/or the second time period of suspected arousal or
analyze the received acoustic data associated with the determined first time section to identify a location of obstruction associated with the user if the pSDB status is indicative of the user having pSDB or
analyze the received acoustic data to further determine or confirm the suspected arousal.

30-36. (canceled)

37. The system of claim 1, wherein the control system is further configured to:
analyze the airflow data to identify (i) one or more sleep stages of the user and (ii) the one or more respiratory events experienced by the user; and
correlate the one or more sleep stages of the user and the one or more respiratory events experienced by the user to determine whether the user experiences is rapid eye movement (REM)-dominant respiratory events.

38. The system of claim 37, wherein the control system is further configured to:
analyze the airflow data associated with the determined first time section to further identify a sleep stage of the user; and
discard the airflow data associated with the determined first time section and when the identified sleep stage of the user is REM sleep.

39-40. (canceled)

41. The system of claim 1, wherein the analyzing the airflow data includes detecting a breathing waveform, which breathing waveform includes an inspiratory waveform and/or an expiratory waveform, and determining a deviation from a normal breathing waveform.

42-49. (canceled)

50. The system of claim 1, wherein the control system is further configured to:
provide control signals to the respiratory device; and
responsive to the pSDB status, determine a modification to pressure settings of the respiratory device, the pressure settings being associated with pressurized air supplied to the airway of the user.

51. The system of claim 1, wherein the control system is further configured to:
provide control signals to a smart pillow; and
responsive to the pSDB status, determine a modification to the smart pillow such that implementation of the modification to the smart pillow urges the user to change position of the user's head.

52. The system of claim 1, wherein the control system is further configured to:
provide control signals to a smart bed or a smart mattress; and
responsive to the pSDB status, determine a modification to the smart bed or the smart mattress such that implementation of the modification to the smart bed or the smart mattress urges the user to change position of the user's body.

53. The system of claim 1, wherein the control system is further configured to:
provide control signals to a wearable device, the wearable device being couplable to a body part of the user; and
responsive to the pSDB status, determine a modification to the wearable device such that implementation of the modification to the wearable device stimulates the user to change position of the user's body.

54-62. (canceled)

63. A non-transitory computer readable medium storing instructions which, when executed by a computer, cause the computer to carry out the operations of:
receiving airflow data associated with a user of the respiratory device;
analyzing the airflow data to identify a first time period of suspected arousal and a second time period of suspected arousal;
determining a first time section between the identified first time period and the identified second time period;
analyzing the airflow data associated with the determined first time section to identify (i) an indication of one or more respiratory events, (ii) an indication of one or more therapy events, or (iii) both (i) and (ii); and
based at least in part on the (i) identified indication of one or more respiratory events, (ii) identified indication of one or more therapy events, or (iii) both (i) and (ii), determining the positional sleep disordered breathing (pSDB) status of the user, the pSDB status being indicative of whether or not the user has pSDB.

64. (canceled)

65. A system for determining a positional sleep disordered breathing (pSDB) status associated with a user, the system including a control system configured to:
receive sensor data associated with the user;
analyze the sensor data to identify a first time period of suspected arousal and a second time period of suspected arousal;
determine a first time section between the identified first time period and the identified second time period;
analyze the sensor data associated with the determined first time section to identify an indication of one or more respiratory events; and
based at least in part on the identified indication of one or more respiratory events, determine the pSDB status of the user, the pSDB status being indicative of whether or not the user has pSDB.

66-121. (canceled)
